(19) United States
(12) Patent Application Publication
(10) Pub. No.: US 2022/0195496 A1
(43) Pub. Date: Jun. 23, 2022

(54) SEQUENCING MICROBIAL CELL-FREE DNA FROM ASYMPTOMATIC INDIVIDUALS

(71) Applicant: Karius, Inc., Redwood City, CA (US)

(72) Inventors: Asim AHMED, Redwood City, CA (US); Radha DUTTAGUPTA, Redwood City, CA (US)

(21) Appl. No.: 17/553,583

(22) Filed: Dec. 16, 2021

Related U.S. Application Data
(60) Provisional application No. 63/127,039, filed on Dec. 17, 2020.
(60) Provisional application No. 63/139,243, filed on Jan. 19, 2021.

(51) Int. Cl. C12Q 1/689; C12Q 1/6895; C12N 15/10

(52) U.S. Cl. CPC C12Q 1/689; C12N 15/1065; C12Q 1/6895

(57) ABSTRACT

Disclosed herein are methods of detecting and treating subjects for bloodstream infections (BSI). Disclosed herein are methods of predicting a bloodstream infection prior to an onset of a symptom. Disclosed herein are method of detecting bloodstream infections using high-throughput sequencing of microbial cell free nucleic acids.

### CROSS-REFERENCE

This application claims the benefit of U.S. Provisional Patent Application 63/127,039 filed on Dec. 17, 2020, and U.S. Provisional Patent Application 63/139,243 filed on Jan. 19, 2021, both of which applications are incorporated herein by reference in their entireties.

### BACKGROUND OF THE INVENTION

Serious infections, especially bloodstream infections (BSIs), are among the most important complications affecting patients receiving treatment for cancer. An incident of BSI-related sepsis can cause death, multiorgan failure, or neurocognitive damage. Although a predictive test that enables preemptive, pathogen-directed therapy could reduce BSI related morbidity and mortality, validated testing appears not to be available.

Invasive fungal infections (IFIs) are also a life-threatening complication of cancer therapy or hematopoietic cell transplantation (HCT). Diagnosis of IFI can be invasive and challenging, and IFI has poor outcomes. Prediction or early non-invasive diagnosis of IFI in high-risk hosts before onset of symptoms could reduce morbidity and mortality. There is a need for non-invasive assays that can predict BSIs and IFIs.

### SUMMARY OF THE INVENTION

Disclosed herein in some embodiments, is a method of treating a subject at risk for a bloodstream infection comprising: collecting one or more blood samples from the subject, wherein the one or more blood samples comprise microbial cell-free nucleic acids (mcfNA) and the subject is afebrile or blood culture negative; detecting an amount of a pathogen associated with the bloodstream infection based on the microbial cell-free nucleic acids (mcfNA) in the one or more blood samples; predicting that the subject at risk for the bloodstream infection will experience a symptom of a bloodstream infection or become blood-culture positive for the bloodstream infection based on the amount of the pathogen associated with the bloodstream infection, wherein the predicting has a predictive sensitivity of at least 50%, at least 55%, at least 60%, at least 65%, at least 70%, at least 75%, at least 80%, at least 85%, at least 90%, or at least 95% for samples collected at least three days prior to onset of a blood-culture positive infection or onset of symptoms of a bloodstream infection; and administering a therapeutic treatment to the subject prior to onset of the symptom of the bloodstream infection or a sign of the bloodstream infection. In some embodiments, the pathogen associated with the bloodstream infection is a bacterium, and the bloodstream infection is a bacterial bloodstream infection. In some embodiments, the pathogen associated with the bloodstream infection is at least one pathogen selected from the group consisting of: a Bacillus spp, Clostridium spp., Corynebacterium jeikeium, Enterococcus spp., Rothia spp., Lactobacillus spp., Streptococcus spp., Staphylococcus spp, Citrobacter spp., Escherichia coli, Klebsiella spp., Pseudomonas spp., Stenotrophomonas maltophilia, and Candida spp. In some embodiments, the pathogen associated with the bloodstream infection is at least one pathogen selected from the group consisting of: Bacillus spp, Clostridium spp., Corynebacterium jeikeium, Lactobacillus spp., Staphylococcus spp, Citrobacter spp., Klebsiella spp., Pseudomonas spp., Stenotrophomonas maltophilia, and Candida spp. In some embodiments, the pathogen associated with the bloodstream infection is a fungus and the bloodstream infection is a fungal infection. In some embodiments, the fungus is a Candida spp. In some embodiments, the fungus is a Candida krusei. In some embodiments, the mcfNA are microbial cell-free DNA. In some embodiments, the one or more blood samples are one or more plasma samples. In some embodiments, the method can further comprise attaching the mcfNA to adapters in order to prepare a sequencing library. In some embodiments, the method can further comprise generating sequence reads from the mcfNA, aligning the sequence reads to bacterial or fungal DNA sequences in a reference data set to obtain aligned sequence reads, and identifying the pathogen associated with the bloodstream infection based on the aligned sequence reads. In some embodiments, the method can further comprise generating sequence reads from the mcfNA, aligning the sequence reads to bacterial or fungal nucleic acid sequences in a reference data set to obtain aligned sequence reads, and identifying the pathogen associated with the bloodstream infection based on the aligned sequence reads. In some embodiments, the predictive sensitivity of at least 50%, at least 55%, at least 60%, at least 65%, at least 70%, at least 75%, at least 80%, at least 85%, at least 90%, or at least 95% is a predictive sensitivity of at least 50%, at least 55%, at least 60%, at least 65%, at least 70%, at least 75%, at least 80%, at least 85%, at least 90%, or at least 95% for a bacterial bloodstream infection. In some embodiments, the method can further comprise a predictive specificity of greater than at least 50%, at least 55%, at least 60%, at least 65%, at least 70%, at least 75%, at least 80%, at least 85%, at least 90%, or at least 95% for predicting bloodstream infection associated with at least one pathogen selected from the group consisting of: Bacillus spp, Clostridium spp., Corynebacterium jeikeium, Enterococcus spp., Rothia spp., Lactobacillus spp., Staphylococcus spp, Citrobacter spp., Escherichia coli, Klebsiella spp., Pseudomonas spp., Stenotrophomonas maltophilia, and Candida spp. In some cases, the predictive sensitivity for detecting a bacterial infection is for samples collected within 1,2, 3, 4, 5, 6, 7, 8, 9, or 10 days prior to onset of a sign (e.g., blood-culture negative infection) or symptom of a bacterial bloodstream infection. In some cases, the predictive sensitivity for detecting a fungal infection is for samples collected within 1,2, 3, 4, 5, 6, 7, 8, 9, 10, 12, 15, 20, 25, or 30 days prior to onset of a sign (e.g., blood-culture negative infection) or symptom of a fungal bloodstream infection. In some embodiments, the one or more blood samples comprise at least two blood samples collected prior to onset of infection and within a 7 day period; or at least three blood samples collected prior to onset of the bloodstream infection and within a 7 day period. In some embodiments, the method can further comprise identifying a second pathogen associated with the bloodstream infection. In some embodiments, the therapeutic treatment is a pathogen-directed therapy. In some embodiments, the pathogen-directed therapy is at least one therapy selected from the group consisting of: vancomycin, ampicillin, cefepime, penicillin, meropenem, ceftriaxone, levofloxacin, and linezolid. In some embodiments, the subject has no infection within seven days prior to collecting the one or more blood samples. In some embodiments, the method can further comprise spiking the one or more plasma samples with a known concentration of synthetic DNA. In some embodiments, a concentration of the bacterial or fungal mcfNA per microliter of blood is measured. the concentration of bacterial or fungal mcfNA per microliter of blood is greater than a threshold amount. In some embodiments, the subject does not have any symptoms associated with the bloodstream infection. In some embodiments, the the subject is not neutropenic. In some embodiments, the subject is blood culture negative during the collecting of the one or more blood samples. In some embodiments, the bloodstream infection is a gram-negative bacterial infection. In some embodiments, the bloodstream infection is a gram-positive bacterial infection. In some embodiments, the bloodstream infection is susceptible to empirical antimicrobial therapy. In some embodiments, the concentration of bacterial or fungal mcfDNA is at least 100, at least 200, at least 300, at least 400, at least 500, at least 600, at least 700, at least 800, at least 900, or at least 1000 molecules per microliter (MPM) of plasma. In some embodiments, the concentration of bacterial mcfDNA is at least 1,000 molecules per microliter (MPM) of plasma. In some embodiments, the detecting the amount of the pathogen associated with the bloodstream infection comprises detecting the pathogen associated with the bloodstream infection at a strain or species level.

Disclosed herein in some embodiments, is a method of detecting or predicting infection, or symptoms of an infection, in a subject at risk for a bloodstream infection comprising: collecting one or more blood samples from the subject, wherein the one or more blood samples comprise microbial cell-free nucleic acids (mcfNA) and the subject is afebrile; detecting an amount of a pathogen associated with the bloodstream infection based on the microbial cell-free nucleic acids (mcfNA) in the one or more blood samples; and predicting that the subject at risk for the bloodstream infection will experience a symptom of a bloodstream infection based on the amount of the pathogen associated with the bloodstream infection, wherein the predicting has a predictive sensitivity of at least 50%, at least 55%, at least 60%, at least 65%, at least 70%, at least 75%, at least 80%, at least 85%, at least 90%, or at least 95% for samples collected at least three days prior to onset of symptoms of a bloodstream infection or prior to onset of a blood-culture positive infection. In some embodiments, the pathogen associated with the bloodstream infection is a bacterium, and the bloodstream infection is a bacterial bloodstream infection. In some embodiments, the pathogen associated with the bloodstream infection is at least one pathogen selected from the group consisting of: a Bacillus spp, Clostridium spp., Corynebacterium jeikeium, Enterococcus spp., Rothia spp., Lactobacillus spp., Streptococcus spp., Staphylococcus spp, Citrobacter spp., Escherichia coli, Klebsiella spp., Pseudomonas spp., Stenotrophomonas maltophilia, and Candida spp. In some embodiments, the pathogen associated with the bloodstream infection is at least one pathogen selected from the group consisting of: Bacillus spp, Clostridium spp., Corynebacterium jeikeium, Lactobacillus spp., Staphylococcus spp, Citrobacter spp., Klebsiella spp., Pseudomonas spp., Stenotrophomonas maltophilia, and Candida spp. In some embodiments, the pathogen associated with the bloodstream infection is a fungus and the bloodstream infection is a fungal infection. In some embodiments, the fungus is a Candida spp. In some embodiments, the fungus is a Candida krusei. In some embodiments, the mcfNA are microbial cell-free DNA. In some embodiments, the one or more blood samples are one or more plasma samples. In some embodiments, the method can further comprise attaching the mcfNA to adapters in order to prepare a sequencing library. In some embodiments, the method can further comprise generating sequence reads from the mcfNA, aligning the sequence reads to bacterial or fungal DNA sequences in a reference data set to obtain aligned sequence reads, and identifying the pathogen associated with the bloodstream infection based on the aligned sequence reads. In some embodiments, the method can further comprise generating sequence reads from the mcfNA, aligning the sequence reads to bacterial or fungal nucleic acid sequences in a reference data set to obtain aligned sequence reads, and identifying the pathogen associated with the bloodstream infection based on the aligned sequence reads. In some embodiments, the predictive sensitivity of at least 50%, at least 55%, at least 60%, at least 65%, at least 70%, at least 75%, at least 80%, at least 85%, at least 90%, or at least 95% is a predictive sensitivity of at least 50%, at least 55%, at least 60%, at least 65%, at least 70%, at least 75%, at least 80%, at least 85%, at least 90%, or at least 95% for a bacterial bloodstream infection. In some embodiments, the method can further comprise a predictive specificity of greater than at least 50%, at least 55%, at least 60%, at least 65%, at least 70%, at least 75%, at least 80%, at least 85%, at least 90%, or at least 95% for predicting bloodstream infection associated with at least one pathogen selected from the group consisting of: Bacillus spp, Clostridium spp., Corynebacterium jeikeium, Enterococcus spp., Rothia spp., Lactobacillus spp., Staphylococcus spp, Citrobacter spp., Escherichia coli, Klebsiella spp., Pseudomonas spp., Stenotrophomonas maltophilia, and Candida spp. In some embodiments, the one or more blood samples comprise at least two blood samples collected prior to onset of infection and within a 7 day period; or at least three blood samples collected prior to onset of the bloodstream infection and within a 7 day period. In some embodiments, the method can further comprise identifying a second pathogen associated with the bloodstream infection. In some embodiments, the subject has no infection within seven days prior to collecting the one or more blood samples. In some embodiments, the method can further comprise spiking the one or more plasma samples with a known concentration of synthetic DNA. In some embodiments, a concentration of the bacterial or fungal mcfNA per microliter of blood is measured. the concentration of bacterial or fungal mcfNA per microliter of blood is greater than a threshold amount. In some embodiments, the subject does not have any symptoms associated with the bloodstream infection. In some embodiments, the the subject is not neutropenic. In some embodiments, the subject is blood culture negative during the collecting of the one or more blood samples. In some embodiments, the bloodstream infection is a gram-negative bacterial infection. In some embodiments, the bloodstream infection is a gram-positive bacterial infection. In some embodiments, the bloodstream infection is susceptible to empirical antimicrobial therapy. In some embodiments, the concentration of bacterial or fungal mcfDNA is at least 100, at least 200, at least 300, at least 400, at least 500, at least 600, at least 700, at least 800, at least 900, or at least 1000 molecules per microliter (MPM) of plasma. In some embodiments, the concentration of bacterial mcfDNA is at least 1,000 molecules per microliter (MPM) of plasma. In some embodiments, the detecting the amount of the pathogen associated with the bloodstream infection comprises detecting the pathogen associated with the bloodstream infection at a strain or species level.

Disclosed herein in some embodiments, is a method of processing and analyzing a blood sample from a blood cancer patient who is asymptomatic for an infection by a bacterial or fungal organism comprising: preparing at least one plasma sample comprising microbial cell-free nucleic acids (mcfNA) from the blood cancer patient asymptomatic for the infection by the bacterial or fungal organism; preparing a first library comprising the mcfNA attached to adapters; subjecting the first library comprising the mcfNA attached to the adapters to next generation sequencing to produce sequence reads; aligning the sequence reads to bacterial or fungal DNA sequences in a reference data set to obtain aligned sequence reads; and detecting a presence of and quantifying the bacterial or fungal organism at a species or strain level based on the aligned sequence reads. In some embodiments, the the microbial cell-free nucleic acids comprise microbial cell-free DNA. In some embodiments, the method can further comprise preparing a second microbial cell-free nucleic acid library comprising microbial-cell free nucleic acids obtained from a second blood cancer patient. In some embodiments, the method can further comprise multiplexing the first and second microbial cell-free nucleic acid libraries and subjecting the first and second microbial cell-free nucleic acid libraries in the multiplex to the next generation sequencing. In some embodiments, the method can further comprise subjecting the at least one plasma sample to high-speed centrifugation. In some embodiments, the high-speed centrifugation comprises centrifuging the at least one plasma sample to at least about 16,000 rcf. In some embodiments, the method can further comprise preparing the at least one plasma sample from a blood sample from at least one blood cancer patient. In some embodiments, the method can further comprise freezing the at least one plasma sample. In some embodiments, the method can further comprise spiking the at least one plasma sample with a known concentration of synthetic DNA molecules. In some embodiments, the blood cancer is a leukemia. In some embodiments, the blood cancer is acute myeloid leukemia (AML) or acute lymphoblastic leukemia (ALL). In some embodiments, the patient is receiving chemotherapy. In some embodiments, the patient is the recipient of a hematopoietic stem cell transplant. In some embodiments, the patient is immunocompromised. In some embodiments, the patient is a pediatric patient. In some embodiments, the patient is not neutropenic. In some embodiments, the patient is blood culture negative. In some embodiments, the method can further comprise administering an antimicrobial treatment to the cancer patient prior to the onset of the infection by a bacterial or fungal organism. In some embodiments, the infection by a bacterial or fungal organism is a bacterial bloodstream infection. In some embodiments, the infection by a bacterial or fungal organism is identified at a strain or species level at least three days prior to onset of the bacterial bloodstream infection. In some cases, onset of the infection refers to onset of a blood-culture positive infection. In some embodiments, the infection by a bacterial or fungal organism is an invasive fungal infection. In some embodiments, the infection by a bacterial or fungal organism at the species or strain level is identified at least ten days prior to onset of the invasive fungal bloodstream infection.

Disclosed herein in some embodiments, is a method of processing and analyzing a blood sample from a cancer patient at risk of a bacterial or fungal infection comprising: preparing plasma samples comprising microbial cell-free DNA (mcfDNA) from at least two longitudinal blood samples collected from the cancer patient within seven days prior to the onset of a bloodstream infection; isolating the mcfDNA from the plasma samples; attaching adapters to the mcfDNA to produce a DNA library comprising mcfDNA attached to the adapters; obtaining sequence reads from the DNA library; aligning the sequence reads to bacterial or fungal DNA sequences in a reference data set to obtain aligned sequence reads; identifying the infection by a bacterial or fungal organism based on the aligned sequence reads; and quantifying the mcfDNA at the species or strain level prior to onset of the infection by a bacterial or fungal organism. In some embodiments, the at least two longitudinal blood samples comprise at least three longitudinal blood samples collected from the cancer patient within at least five days. In some embodiments, the at least two longitudinal blood samples are collected within a seven-day period. In some embodiments, the infection by a bacterial or fungal organism is identified in at the least two successive longitudinal blood samples. In some embodiments, the infection by a bacterial or fungal organism is identified within 28 hours after the blood sample is collected. In some embodiments, the cancer patient has no infection within seven days prior to collecting the at least two longitudinal blood samples. In some embodiments, the method can further comprise administering an antimicrobial therapeutic treatment to the cancer patient prior to onset of the bacterial or fungal bloodstream infection. In some embodiments, the method can further comprise spiking each of the plasma samples with a known concentration of synthetic DNA. In some embodiments, the concentration of the bacterial or fungal mcfDNA per microliter of blood is measured. In some embodiments, the concentration of bacterial or fungal mcfDNA per microliter of blood is greater than a threshold amount. In some embodiments, the patient is afebrile or asymptomatic. In some embodiments, the patient is not neutropenic. In some embodiments, the patient is blood culture negative. In some embodiments, the bacterial or fungal infection is associated with a pathogen that is at least one pathogen selected from the group consisting of Bacillus spp., Clostridium spp, Corynebactehum jeikeium, Enterococcus spp., Lactobacillus spp., Rothia spp., Staphylococcus spp., Streptococcus spp., Citrobacter spp., Escherichia coli, Klebsiella spp., Pseudomonas spp., Stenotrophomonas maltophilia, and Candida spp. In some embodiments, the bacterial or fungal infection is a bacterial infection. In some embodiments, the bacterial infection is a gram-negative bacterial infection. In some embodiments, the bacterial infection is a gram-positive bacterial infection. In some embodiments, the bacterial or fungal infection is a fungal infection.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The patent application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the sensitivity of mcfDNA-seq for the prediction or diagnosis of BSI by day before the onset of infection. Logical derivation was used to impute values for missing data. BSI indicates bloodstream infection; mcfDNA-seq, plasma microbial cell-free DNA sequencing. Error bars show 95% CIs. Overall specificity of mcfDNA-seq was 82% (95% CI, 66%-91%), and specificity for common BSI pathogens was 91% (95% CI, 76%-97%).

FIG. 2 shows the population kinetics of pathogen DNA by day before the onset of BSI. Circles represent individual values, lines represent penalized B-spline smoothing curves for bloodstream infection (BSI) episodes, and bands represent 95% CIs. Orange dots indicate a gram-negative pathogen; dark blue dots, a gram-positive pathogen; brown dots, overlapping samples.

FIG. 3 shows the method of selection of plasma samples for testing as negative controls and bloodstream infection (BSI) episodes. Closed shapes represent available samples for an individual participant; negative control samples were defined as plasma samples obtained when no fever or infection was documented in the prior or subsequent 7 days; BSI samples were defined as plasma samples obtained in the 7 days before or 4 days after the onset of BSI. Samples obtained on the day of onset of BSI were classified as diagnostic samples; samples obtained on the 3 days before the onset of BSI were classified as predictive samples.

FIG. 4 shows the method of logical derivation for imputation of missing mcfDNA-seq results.

FIG. 5 shows the sensitivity of mcfDNA-seq by day prior to bloodstream infection using raw data without imputation.

FIGS. 6A-6D show the sensitivity of predictive mcfDNA-seq for bacterial BSI by frequency of testing. For each frequency of testing model, estimated sensitivities for event in each day of the week were plotted in a radar graph. Larger shaded area indicates higher overall sensitivity. The red line represents favorable sensitivity of 50%. Logical derivation was used to impute values for missing data. BSI, bloodstream infection; mcfDNA-seq, plasma microbial cell-free DNA sequencing. FIG. 6A shows the sensitivity for tests on Mondays. FIG. 6B shows the sensitivity for tests on Mondays and Thursdays. FIG. 6C shows the sensitivity for tests on Mondays, Wednesday, and Fridays. FIG. 6D shows the sensitivity for tests on Monday through Friday. Testing thrice weekly was required to keep minimum sensitivity above 50%.

FIGS. 7A-7D show the sensitivity of predictive mcfDNA-seq for bacterial BSI by frequency of testing using raw data without imputation. For each frequency of testing model, estimated sensitivities for event in each day of the week were plotted in a radar graph. Larger shaded area indicates higher overall sensitivity. BSI, bloodstream infection; mcfDNA-seq, plasma microbial cell-free DNA sequencing. FIG. 7A shows the sensitivity for tests on Mondays. FIG. 7B shows the sensitivity for tests on Mondays and Thursdays. FIG. 7C shows the sensitivity for tests on Mondays, Wednesday, and Fridays. FIG. 7D shows the sensitivity for tests on Monday through Friday.

FIG. 8A shows Mucor velutinosus DNA kinetics in samples collected prior to invasive fungal infection in a child (PQ118) with leukemia. Pathogen DNA concentration is given in molecules of fungus per microliter of plasma (MPM) vs. days relative to clinical diagnosis of invasive sinusitis. Also given is a single measurement of a non-infective fungus, Aureobosidium pullulans, present in, at day −15 the sample. FIG. 8B shows Aspergillus flavus/oryzae DNA kinetics in samples collected prior to invasive pulmonary Aspergillosis infection in a child (PQ123) with leukemia. Pathogen DNA concentration is given in molecules of fungus per microliter of plasma (MPM) vs. days relative to clinical diagnosis of an invasive pulmonary infection. Also given is a single measurement of a non-infective fungus, Malassezia globosa, present in the sample at day −12. FIG. 8C shows Histoplasma capsulatum DNA kinetics in samples collected prior to invasive disseminated Histoplasmosis infection in a child (PQ107) with leukemia. Pathogen DNA concentration is given in molecules of fungus per microliter of plasma (MPM) vs. days relative to clinical diagnosis of invasive infection. Also given are single measurements of a non-infective fungus, Malassezia globosa, present in the sample at day −20 and Penicillium decumbens, present in the sample at day −6. FIG. 8D shows Aspergillus flavus/oryzae DNA kinetics in samples collected prior to invasive pulmonary Aspergillus spp. invasive gingivitis infection in a child (PQ120) with leukemia. Pathogen DNA concentration is given in molecules of fungus per microliter of plasma (MPM) vs. days relative to clinical diagnosis of invasive gingivitis.

### DETAILED DESCRIPTION

Overview

Disclosed herein, in some embodiments, are methods for detecting microbial cell-free nucleic acids (e.g., microbial cell-free DNA “mcfDNA”) in a subject (e.g., patient) who does not have symptoms of an infection or who is blood culture-negative for infection in order to determine or predict whether the subject will later experience a sign or symptom of an infection such as a bloodstream infection (e.g., bacterial bloodstream infection, fungal bloodstream infection) or invasive fungal infection (e.g., fungal infection at a site such as paranasal sinus, pulmonary, gingiva or disseminated fungal infection). In some cases, a sign of infection occurs when the infection is blood-culture positive. Generally, the methods provided herein involve detection and/or quantification of microbial cell free nucleic acids (e.g., microbial cell-free DNA, microbial cell-free RNA) in a sample from a subject (e.g., plasma). In some cases, the sample (e.g., plasma sample) is spiked with a known concentration of synthetic DNA for quality control purposes. The methods can comprise attaching a nucleic acid adapter (e.g., DNA adapter) to the cell-free nucleic acids (e.g., cell-free DNA) and preparing a sequencing library. In some cases, the methods comprise attaching a first adapter to DNA from a first subject and a second adapter comprising a different sequence to a DNA sample from a second subject to produce first and second DNA libraries respectively. In some cases, the first and second DNA libraries are combined. The libraries may be subjected to multiplex sequencing (e.g., next generation sequencing, metagenomic sequencing), after which the sequence reads are demultiplexed. In some cases, the sequencing comprises performing sequencing-by-synthesis reactions using reversible terminators, particularly fluorescently-labeled reversible terminators (e.g., fluorescently-labeled ddNTP, dNTP). In some embodiments, sequence reads exhibiting strong alignment against human references or the synthetic molecule references are excluded from the analysis. In some cases, sequence reads are filtered based on sequencing quality. In some embodiments, the remaining reads are aligned against a microorganism database. In some embodiments, an expectation maximization algorithm is applied to compute the maximum likelihood estimate of each taxon abundance. In some cases, quantity of each organism is expressed as Molecules per Microliter (MPM), which can refer to the number of DNA sequence reads from the reported organism present per microliter of plasma. In some embodiments, the methods provided herein have a predictive sensitivity of greater than 75% (e.g., greater than 80%, greater than 90%, greater than 95%, greater than 97%) for predicting or detecting an infection (e.g., bacterial infection, bacterial BSI, fungal infection, fungal BSI, IFI) at least 1, 2, 3, 4, 5, 6, 7, 10, 12, 14, 15, 20, 30 or more days prior to onset of infection or onset of symptoms of such infection. In some cases, the prediction or detection of the infection (or symptoms thereof) occurs at a strain or species level. In some cases, the prediction or detection of the infection (or symptoms thereof) occurs at a genus level or family level. In some embodiments, the method further comprises treating the subject for the infection, such as administering a treatment prior to onset of infection, particularly, prior to onset of symptoms of infection. In some embodiments, the treating is prophylactic or preemptive and prevents progression or onset of the fungal or bacterial infection. In some cases, the treatment is an antimicrobial treatment (e.g., antibiotic or antifungal drug). In some cases, the treatment is a broad-spectrum drug. In some cases, the treatment specifically targets a particular microbe.

The methods provided herein generally have the advantage of being rapid and non-invasive. In some cases, the process from DNA extraction to analysis is completed in at most 20 hours, at most 24 hours, at most 28 hours, at most 30 hours, at most 36 hours, or at most 48 hours. In addition, the methods provided herein can help a patient preemptively treat an infection and therefore avoid experiencing symptoms of an infection, along with its associated dangers and risks. In addition, the methods can be particularly useful for certain patient populations susceptible to serious infections such as sepsis, including patients with blood cancer (e.g., leukemia, acute leukemia, acute myeloid leukemia (AML), acute lymphoblastic leukemia (ALL)) who may or may not also be undergoing cancer treatment.

In the present disclosure, wherever aspects are described herein with the language “comprising,” otherwise analogous aspects described in terms of “consisting of” and/or “consisting essentially of” are also provided. All definitions herein described whether specifically mentioned or not, should be construed to refer to definitions as used throughout the specification and attached claims.

Numeric ranges are inclusive of the numbers defining the range. The term “about” as used herein generally means plus or minus ten percent (10%) of a value, inclusive of the value, unless otherwise indicated by the context of the usage. For example, “about 100” refers to any number from 90 to 110.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than,” “less than or equal to,” or “at most” applies to each of the numerical values in that series of numerical values. For example, less than or equal to 3, 2, or 1 is equivalent to less than or equal to 3, less than or equal to 2, or less than or equal to 1.

The term “attach” and its grammatical equivalents may refer to connecting two molecules using any mode of attachment. For example, attaching may refer to connecting two molecules by chemical bonds or other method to generate a new molecule. Attaching an adapter to a nucleic acid may refer to forming a chemical bond between the adapter and the nucleic acid. In some cases, attaching is performed by ligation, e.g., using a ligase. For example, a nucleic acid adapter may be attached to a target nucleic acid by ligation, via forming a phosphodiester bond catalyzed by a ligase.

As used herein, the term “or” is used to refer to a nonexclusive or, such as “A or B” includes “A but not B,” “B but not A,” and “A and B,” unless otherwise indicated.

As used herein, “a”, “an”, and “the” can include plural referents unless otherwise limited expressly or by context.

Subjects

The term “subject” as used herein includes patients, particularly human patients. The term “subject” also encompasses mammals, particularly humans.

In some embodiments disclosed herein, a subject is at risk of having an infection (e.g., high risk of having an infection), particularly at the time of collecting a sample from the subject. As used herein, a subject with a “high risk” of experiencing a bloodstream infection or invasive fungal infection is a subject with a risk that is higher than that of a healthy subject. For example, a patient who is immunocompromised is generally at high risk of experiencing a bloodstream infection or IFI when compared to a healthy patient who is not immunocompromised.

In some embodiments, a subject has an infection, particularly at the time of collection of a sample from the subject. In some cases, the subject is at risk of developing in the future one or more symptoms of infection. In some embodiments, the subject has no sign of an infection. In some embodiments, the subject is blood-culture negative at the time of collection of a sample. In some embodiments, the subject is blood-culture positive at the time of collection of a sample. In some embodiments, the subject is blood-culture positive at the time of collection of a sample for one or more pathogens and blood culture negative for one or more pathogens that later develop into an infection. In some cases, the subject is blood culture negative for a microbe or pathogen detected or predicted by the methods provided herein at the time of collection of the sample. In some embodiments, a subject has no symptoms of infection at the time of collection of a sample or samples from the subject. In cases, the subject has had no sign of infection at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days prior to collection of a sample from the subject. In some embodiments, a symptom of an infection includes a fever, chills, elevated temperature, fatigue, a cough, congestion, fever, elevated heart rate, low blood pressure, hyperventilation, a sore throat, or any combination thereof In some embodiments, a fever is a rectal, ear or temporal artery temperature of 100.4° F. (38° C.) or higher, an oral temperature of 100° F. (37.8° C.) or higher, an armpit temperature of 99° F. (37.2° C.) or higher, or any combination thereof. In some embodiments, a sample is collected and analyzed to determine a risk of a future infection or the risk of developing symptoms of infection in the future.

In some embodiments, the subject is a child. In some embodiments, a child is less than about 18 years of age. In some embodiments, the subject is a pediatric patient. In some embodiments, a subject is an adult. In some embodiments, a subject is less than about 25 years of age. In some embodiments, a subject is elderly. In some embodiments a subject is more than 65 years of age. In some cases, the subject has a high risk of experiencing a bloodstream infection or invasive fungal infection.

In some embodiments, the subject has, is suspected of having, or is at risk of having an infection by a bacterium, a fungus, a virus, a parasite, or any combination thereof, or symptoms of such infection. In some embodiments, the infection by a bacterium, a fungus, a virus, a parasite, or any combination thereof is a bloodstream infection (BSI) (e.g., bacterial BSI, fungal BSI). In some embodiments, the infection is a fungal infection (e.g., invasive fungal infection)). In some embodiments, the infection is a bacterial infection (e.g., sepsis, bacterial BSI). In some embodiments, a bacterial or fungal infection can comprise an infection by an organism selected from the group consisting of Bacillus spp., Clostridium spp, Corynebactehum jeikeium, Enterococcus spp., Lactobacillus spp., Rothia spp., Staphylococcus spp., Streptococcus spp., Citrobacter spp., Escherichia coli, Klebsiella spp., Pseudomonas spp., Stenotrophomonas maltophilia, and Candida spp. In some embodiments the microbe or organism is at least one microbe or organism provided in Tables 2-4, and Tables 6-8, or mentioned in the Examples section of this application. In some embodiments, the bacterial infection is a gram-negative bacterial infection. In some embodiments, the bacterial infection is a gram-positive bacterial infection. In some embodiments, the bacterial or fungal infection is susceptible to empirical antimicrobial therapy. In some embodiments, the subject is diagnosed with having an infection or predicted to be at risk of an infection using methods disclosed herein. In some embodiments, a subject is predicted to be at risk of having an infection or at risk of developing symptoms of infection using methods disclosed herein.

A subject can be healthy; or, in some embodiments, the subject has a disease (e.g., cancer, infection). In some embodiments, the subject has cancer. In some embodiments, the cancer is a relapsed or refractory cancer. In some embodiments, the cancer is a blood cancer (e.g., leukemia, chronic leukemia, acute leukemia). In some embodiments, the acute leukemia is acute myeloid leukemia (AML), acute lymphoblastic leukemia (ALL), or a combination thereof In some embodiments, the leukemia comprises a Chronic Lymphocytic Leukemia (CLL), a Chronic Myeloid Leukemia (CML), a Hairy Cell Leukemia, a Chronic Myelomonocytic Leukemia, a Juvenile Myelomonocytic Leukemia, a Large Granular Lymphocytic Leukemia, a Blastic Plasmacytoid Dendritic Cell Neoplasm, a B-Cell Prolymphocytic Leukemia (B-PLL), a T-Cell Prolymphocytic Leukemia (T-PLL), or any combination thereof. In some embodiments, a subject has relapsed or refractory blood cancer (e.g., relapsed/refractory acute leukemia, relapsed/refractory AML, relapsed/refractory ALL).

In some embodiments, the subject is receiving chemotherapy, targeted therapy, immunotherapy, or a combination thereof. In some embodiments, a chemotherapy can comprise an alkylating agent, an antimetabolite, an anti-tumor antibiotic, a topoisomerase inhibitor, a mitotic inhibitor, or any combination thereof. In some embodiments, a subject can be immunosuppressed, e.g., as a result of the chemotherapy. In some embodiments, a subject is a recipient of a hematopoietic stem cell transplant. In some embodiments, the subject has neutropenia. In some embodiments, the subject does not have neutropenia.

### Samples

In some embodiments, a sample is collected from a subject (e.g., a patient). In some embodiments, the sample is a biological sample. In some embodiments, a biological sample is a whole blood sample. In some embodiments, the sample is a cell-free sample, such as a plasma sample or a cell-free plasma sample. In some embodiments, the sample is a sample of isolated or extracted nucleic acids (e.g., DNA, RNA, cell-free DNA). In some embodiments, the plasma sample is collected by collecting blood through venipuncture. In some embodiments, a specimen is mixed with an additive immediately after collection. In some cases, the additive is an anti-coagulant. In some cases, the additive prevents degradation of nucleic acids. In some cases, the additive is EDTA. In some embodiments, measures can be taken to avoid hemolysis or lipemia. In some embodiments, a sample is processed or unprocessed. In some embodiments, a sample is processed by extracting nucleic acids from a biological sample. In some embodiments, DNA is extracted from a sample. In some embodiments, nucleic acids are not extracted from the sample. In some embodiments, a sample comprises nucleic acids. In some embodiments, a sample consists essentially of nucleic acids.

In some cases, the methods provided herein comprise processing whole blood into a plasma sample. In some embodiments, such processing comprises centrifuging the whole blood in order to separate the plasma from blood cells. In some cases, the method further comprises subjecting the plasma to a second centrifugation, often at a higher speed in order to remove bacterial cells and cellular debris. In some cases, the second centrifugation is at a relative centrifugal force (rcf) of least about 4,000 rcf, at least about 5,000 rcf, at least about 6,000 rcf, at least about 8,000 rcf, at least about 10,000 rcf, at least about 12,000 rcf, at least about 14,000 rcf, at least about 16,000 rcf, or at least about 20,000 rcf.

The sample can be collected at a time point before an onset of a symptom, particularly a symptom of infection (e.g., bloodstream infection, invasive fungal infection) or a sign of infection, such as a positive result by blood-culture. In some embodiments, a time point is at least about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, or about 30 days, before onset of a symptom or sign of a fungal infection. In some embodiments, a time point is at most about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, or about 30 days before onset of a symptom or sign of fungal infection. In some embodiments, a time point is at least about 1 day, about 2 days, about 3 days, about 4 days, about 5 days, about 6 days, about 7 days, about 8 days, about 9 days, or about 10 days before onset of a symptom or sign of a bacterial infection. In some embodiments, a time point is at most about 1 day, about 2 days, about 3 days, about 4 days, about 5 days, about 6 days, about 7 days, about 8 days, about 9 days, or about 10 days before onset of a symptom or sign of bacterial infection. In some embodiments, the time point is within a range, e.g., from 2 to 10 days prior to onset of a symptom or sign of bacterial infection, from 3 to 10 days prior to onset of a symptom or sign of bacterial infection, from 4 to 10 days prior to onset of a symptom or sign of bacterial infection, from 4 to 10 days prior to onset of a symptom or sign of bacterial infection, from 5 to 10 days prior to onset of a sign or symptom of bacterial infection, from 6 to 10 days prior to onset of a symptom or sign of bacterial infection, from 7 to 10 days prior to onset of a symptom or sign of bacterial infection, from 8 to 10 days prior to onset of a symptom or sign of bacterial infection, from 1 to 3 days prior to onset of a symptom or sign of bacterial infection, from 1 to 4 days prior to onset of a symptom or sign of bacterial infection, from 1 to 5 days prior to onset of a symptom or sign of bacterial infection, from 1 to 6 days prior to onset of a symptom or sign of bacterial infection, from 2 to 8 days prior to onset of a symptom or sign of bacterial infection, from 2 to 9 days prior to onset of a symptom or sign of bacterial infection, from 3 to 5 days prior to onset of a symptom or sign of bacterial infection, from 3 to 7 days prior to onset of a symptom or sign of bacterial infection, from 3 to 8 days prior to onset of a symptom or sign of bacterial infection, from 4 to 8 days prior to onset of a symptom or sign of bacterial infection, from 5 to 7 days prior to onset of a symptom or sign of bacterial infection, or from 7 to 9 days prior to onset of a symptom or sign of bacterial infection. In some embodiments, the time point is within a range, e.g., from 2 to 20 days prior to onset of a symptom or sign of fungal infection, from 3 to 14 days prior to onset of a symptom or sign of fungal infection, from 3 to 20 days prior to onset of a symptom or sign of fungal infection, from 3 to 30 days prior to onset of a symptom or sign of fungal infection, from 2 to 30 days prior to onset of a sign or symptom of fungal infection, from 5 to 20 days prior to onset of a symptom or sign of fungal infection, from 5-30 days prior to onset of a symptom of fungal infection, from 7 to 20 days prior to onset of a symptom sign of fungal infection, or from 7 to 30 days prior to onset of a symptom or sign of fungal infection.

In some embodiments, a plurality of samples is collected over a series of time points. In some embodiments, a plurality of samples is collected to monitor an onset of a disease, to predict onset of a disease, predict onset of a symptom of disease (e.g., onset of a symptom of infection), to monitor or predict a progression of a disease, to detect a response to treatment for the disease or any combination thereof In some embodiments, the plurality of samples is at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples. In some embodiments, at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples are collected before onset of a symptom. In some embodiments, at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples are collected over a period of time. In some embodiments, a plurality of samples are collected on consecutive days. In some embodiments, at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples are collected on consecutive days. In some embodiments, a plurality of samples are collected on alternate days. In some embodiments, at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples can be collected on alternate days. In some embodiments, the collection of samples can be interspersed between days when no sample is collected. In some embodiments, a schedule of sample collection can repeat over a number of days. In some embodiments, a schedule of sample collection can repeat over 2 days, over 3 days, over 4 days, over 5 days, over 6 days, over 7 days, over 8 days, over 9 days, over 10 days, over 11 days, over 12 days, over 13 days, over 14 days, over 15 days, over 16 days, over 17 days, over 18 days, over 19 days, over 20 days, over 21 days, or over 22 days. In some embodiments, a schedule of sample collection can repeat on the same day, collecting multiple samples from a subject throughout the 24 hours.

In some embodiments disclosed herein, is a method of processing and analyzing a blood sample comprising preparing plasma microbial cell-free nucleic acids (mcfNA) from at least two blood samples collected from the same subject within 30 days, 20 days, within 14 days, within 7 days, within 6 days, within 5 days, within 4 days, within 3 days, within 2 days, or within 1 day before onset of an invasive fungal infection (IFI) (or onset of a symptom of IFI). In some embodiments disclosed herein, is a method of processing and analyzing a blood sample comprising preparing plasma microbial cell-free nucleic acids (mcfNA) from at least two blood samples collected from the same subject within 10 days, within 7 days, within 6 days, within 5 days, within 4 days, within 3 days, within 2 days, or within 1 day before an onset of a bloodstream infection (or onset of a symptom of BSI) (e.g., bacterial infection). In some embodiments, plasma samples comprising microbial cell-free nucleic acids (mcfNA) from at least two blood samples comprise at least three longitudinal blood samples collected from a patient who is at high-risk of blood stream infection within a period of a least five days before an onset of infection or onset of a symptom of infection. In some embodiments, at least two blood samples obtained in at least three longitudinal blood samples from a subject who is at high-risk of blood stream infection can be obtained within a period of a least 3 days before an onset of infection or before onset of a symptom of infection.

Often, a sample disclosed herein comprises a target nucleic acid (e.g., target DNA, target RNA). In some embodiments, a target nucleic acid is a cell-free nucleic acid. For example, the sample can comprise microbial cell-free nucleic acids (e.g., mcfDNA) that comprises a microbial target DNA (e.g., mcfDNA derived from a microbe, which can include pathogenic microbes). Exemplary microbes that can be detected by the methods provided herein include bacteria, fungi, parasites, and viruses. In some embodiments, a cell-free nucleic acid is a circulating cell-free nucleic acid. In some embodiments, a cell free nucleic acid can comprise cell-free DNA.

In some embodiments, nucleic acids (e.g., cell-free nucleic acids) are extracted from a sample. In some embodiments, isolated nucleic acids (e.g., extracted DNA) can be used to prepare DNA libraries. In some embodiments, DNA libraries can be prepared by attaching adapters to nucleic acids. In some embodiments, adapters can be used for sequencing of nucleic acids. In some embodiments, nucleic acids can comprise DNA. In some embodiments, nucleic acids containing adapters can be sequenced to obtain sequence reads. In some embodiments, a sample (e.g., a plasma sample comprising mcfDNA) is mixed with adapters prior to extracting nucleic acids or DNA from the sample. In some embodiments, nucleic acids extracted from a sample (e.g., a plasma sample comprising mcfDNA) are attached to adapters following extraction. In some embodiments, sequence reads can be produced through high-throughput sequencing (HTS). In some embodiments, HTS can comprise next-generation sequencing (NGS). In some embodiments, sequence reads can be aligned to sequences in a reference dataset. In some embodiments, sequences can be a bacterial sequence aligned to a reference dataset to obtain an aligned sequence read. In some embodiments, a sequence can be a fungal sequence aligned to a reference dataset to obtain an aligned sequence read. In some embodiments, an aligned bacterial sequence, a fungal sequence or a combination thereof, can be quantified for bacterial sequences or fungal sequences based on aligned sequence reads obtained.

In the methods provided herein, nucleic acids can be isolated. In some embodiments, nucleic acids can be extracted using a liquid extraction. In some embodiments, a liquid extraction can comprise a phenol-chloroform extraction. In some embodiments, a phenol-chloroform extraction can comprise use of Trizol™, DNAzol™, or any combination thereof In some embodiments, nucleic acids can be extracted using centrifugation through selective filters in a column. In some embodiments, nucleic acids can be concentrated or precipitated by known methods, including, by way of example only, centrifugation. In some embodiments, nucleic acids can be bound to a selective membrane (e.g., silica) for the purposes of purification. In some embodiments, nucleic acids can be extracted using commercially available kits (e.g., QIAamp Circulating Nucleic Acid Kit™, Qiagen DNeasy kit™, QIAamp kit™, Qiagen Midi kit™, QIAprep spin kit™, or any combination thereof). Nucleic acids can also be enriched for fragments of a desired length, e.g., fragments which are less than 1000, 500, 400, 300, 200 or 100 base pairs in length. In some embodiments, enrichment based on size can be performed using, e.g., PEG-induced precipitation, an electrophoretic gel or chromatography material (Huber et al. (1993) Nucleic Acids Res. 21:1061-6), gel filtration chromatography, or TSKgel (Kato et al. (1984) J. Biochem, 95:83-86), which publications are hereby incorporated by reference in their entireties for all purposes.

In some embodiments, a nucleic acid sample can be enriched for a target nucleic acid. In some embodiments, a target nucleic acid is a microbial cell-free nucleic acid.

In some embodiments, target (e.g., pathogen, microbial) nucleic acids are enriched relative to background (e.g., subject) nucleic acids in a sample, for example, by electrophoresis, gel electrophoresis, pull-down (e.g., preferentially pulling down target nucleic acids in a pull-down assay by hybridizing them to complementary oligonucleotides conjugated to a label such as a biotin tag and using, for example, avidin or streptavidin attached to a solid support), targeted PCR, or other methods. Examples of enrichment techniques include, but are not limited to: (a) self-hybridization techniques in which a major population in a sample of nucleic acids self-hybridizes more rapidly than a minor population in a sample; (b) depletion of nucleosome-associated DNA from free DNA; (c) removing and/or isolating DNA of specific length intervals; (d) exosome depletion or enrichment; and (e) strategic capture of regions of interest.

In some embodiments, an enriching step can comprise preferentially removing nucleic acids from a sample that are above about 120, about 150, about 200, or about 250 bases in length. In some embodiments, an enriching step comprises preferentially enriching nucleic acids from a sample that are between about 10 bases and about 60 bases in length, between about 10 bases and about 120 bases in length, between about 10 bases and about 150 bases in length, between about 10 bases and about 300 bases in length between about 30 bases and about 60 bases in length, between about 30 bases and about 120 bases in length, between about 30 bases and about 150 bases in length, between about 30 bases and about 200 bases in length, or between about 30 bases and about 300 bases in length. In some embodiments, an enriching step comprises preferentially digesting nucleic acids derived from the host (e.g., subject). In some embodiments, an enriching step comprises preferentially replicating the non-host nucleic acids.

In some embodiments, a nucleic acid library is prepared. In some embodiments, a double-stranded DNA library, a single-stranded DNA library or an RNA library is prepared. A method of preparing a dsDNA library can comprise ligating an adaptor sequence onto one or both ends of a dsDNA fragment. In some cases, the adaptor sequence comprises a primer docking sequence. In some cases, the method further comprises hybridizing a primer to the primer docking sequence and initiating amplification or sequencing of the nucleic acid attached to the adaptor. In some embodiments, the primer or the primer docking sequence comprises at least a portion of an adaptor sequence that couples to a next-generation sequencing platform. In some embodiments, a method can further comprise extension of a hybridized primer to create a duplex, wherein a duplex comprises an original ssDNA fragment and an extended primer strand. In some embodiments, an extended primer strand can be separated from an original ssDNA fragment. In some embodiments, an extended primer strand can be collected, wherein an extended primer strand is a member of an ssDNA library.

In some cases, the library is prepared in an unbiased manner. For example, in some cases, the library is prepared without using a primer that specifically hybridizes to a microbial nucleic acid. For example, in some embodiments, the only amplification performed on the sample involves the use of a primer specific for a sequence of one or more adapters attached to nucleic acids within the sample. In some cases, whole genome amplification is used to prepare the library prior to attachment of the adapters. In some cases, whole genome amplification is not used to prepare the library. In some cases, one or more primers that specifically hybridize to a microbial nucleic acid (e.g., pathogen, viral, fungal, bacterial or parasite nucleic acid) are used to amplify the sample.

In some cases, multiple DNA libraries from different samples (e.g., samples from different patients or subjects) are combined and then subjected to a next generation sequencing assay. In some cases, the libraries are indexed prior to combining in order to track which library corresponds to which sample. Indexing can involve the inclusion of a specific code or bar code in an adapter, e.g., an adapter that is attached to the nucleic acids are to be analyzed. In some cases, the samples comprise a negative control sample or a positive control sample, or both a negative control sample and a positive control sample.

In some embodiments, a length of a nucleic acid can vary. In some embodiments, a nucleic acid or nucleic acid fragment (e.g., dsDNA fragment, RNA, or randomly sized cDNA) can be less than 1000 bp, less than 800 bp, less than 700 bp, less than 600 bp, less than 500 bp, less than 400 bp, less than 300 bp, less than 200 bp, or less than 100 bp. In some embodiments, a DNA fragment can be about 40 to about 100 bp, about 50 to about 125 bp, about 100 to about 200 bp, about 150 to about 400 bp, about 300 to about 500 bp, about 100 to about 500 bp, about 400 to about 700 bp, about 500 to about 800 bp, about 700 to about 900 bp, about 800 to about 1000 bp, or about 100 to about 1000 bp. In some embodiments, a nucleic acid or nucleic acid fragment (e.g., dsDNA fragment, RNA, or randomly sized cDNA) can be within a range from about 20 to about 200 bp, such as within a range from about 40 to about 100 bp.

In some embodiments, an end of a dsDNA fragment can be polished (e.g., blunt-ended) or be subject to end-repair to create a blunt end. In some embodiments, an end of a DNA fragment can be polished by treatment with a polymerase. In some embodiments, a polishing can involve removal of a 3′ overhang, a fill-in of a 5′ overhang, or a combination thereof In some embodiments, a polymerase can be a proof-reading polymerase (e.g., comprising 3′ to 5′ exonuclease activity). In some embodiments, a proofreading polymerase can be, e.g., a T4 DNA polymerase, Pol 1 Klenow fragment, or Pfu polymerase. In some embodiments, a polishing can comprise removal of damaged nucleotides (e.g., abasic sites).

In some embodiments, a ligation of an adaptor to a 3′ end of a nucleic acid fragment can comprise formation of a bond between a 3′ OH group of the fragment and a 5′ phosphate of the adaptor. Therefore, removal of 5′ phosphates from nucleic acid fragments can minimize aberrant ligation of two library members. Accordingly, in some embodiments, 5′ phosphates are removed from nucleic acid fragments. In some embodiments, 5′ phosphates are removed from at least 50%, 55%, 60%, 65%, 70%, 75%, 80%, 85%, 90%, 95%, or greater than 95% of nucleic acid fragments in a sample. In some embodiments, substantially all 5′ phosphate groups are removed from nucleic acid fragments. In some embodiments, substantially all 5′ phosphates are removed from at least 50%, 55%, 60%, 65%, 70%, 75%, 80%, 85%, 90%, 95%, or greater than 95% of nucleic acid fragments in a sample. Removal of 5′ phosphate groups from a nucleic acid sample can be by any means known in the art. Removal of phosphate groups can comprise treating the sample with heat-labile phosphatase. In some embodiments, 5′ phosphate groups are not removed from the nucleic acid sample. In some embodiments, ligation of an adaptor to the 5′ end of the nucleic acid fragment is performed.

Exemplary Sample Processing

What follows are non-limiting examples of methods provided by this disclosure. In some cases, plasma is spiked with a known concentration of synthetic normalization molecule controls. In some cases, the plasma is then subjected to cell-free NA (cfNA) extraction (e.g., extraction of cell-free DNA). The extracted cfNA can be processed by end-repair and ligated to adapters containing specific indexes to end-repaired cfDNA. The products of the ligation can be purified by beads. In some embodiments, the cfDNA ligated to adapters can be amplified with P5 and P7 primers, and the amplified, adapted cfDNA is purified.

Purified cfDNA attached to adapters derived from a plasma sample can be incorporated into a DNA sequencing library. Sequencing libraries from several plasma samples can be pooled with control samples, purified, and, in some embodiments, sequenced on Illumina sequencers using a 75-cycle single-end, dual index sequencing kit. Primary sequencing output can be demultiplexed followed by quality trimming of the reads. In some embodiments, the reads that pass quality filters are aligned against human and synthetic references and then excluded from the analysis, or otherwise set aside. Reads potentially representing human satellite DNA can also filtered, e.g., via a k-mer-based method; then the remaining reads can be aligned with a microorganism reference database, (e.g., a database with 20,963 assemblies of high-quality genomic references). In some embodiments, reads with alignments that exhibit both high percent identity and/or high query coverage can be retained, except, e.g., for reads that are aligned with any mitochondrial or plasmid reference sequences. PCR duplicates can be removed based on their alignments. Relative abundances can be assigned to each taxon in a sample based on the sequencing reads and their alignments.

For each combination of read and taxon, a read sequence probability can be defined that accounts for the divergence between the microorganism present in the sample and the reference assemblies in the database. A mixture model can be used to assign a likelihood to the complete collection of sequencing reads that included the read sequence probabilities and the (unobserved) abundances of each taxon in the sample. In some cases, an expectation-maximization algorithm is applied to compute the maximum likelihood estimate of each taxon abundance. From these abundances, the number of reads arising from each taxon can be aggregated up the taxonomic tree. The estimated taxa abundances from the no template control (NTC) samples within the batch can be combined to parameterize a model of read abundance arising from the environment with variations driven by counting noise. Statistical significance values can then be computed for each estimate of taxon abundance in each patient sample. In some embodiments, taxa that exhibit a high significance level, and are one of the 1449 taxa within the reportable range, comprise the candidate calls. Final calls can be made after additional filtering is applied, which accounts for read location uniformity as well as cross-reactivity risk originating from higher abundance calls. The microorganism calls that pass these filters are reported along with abundances in MPM, as estimated using the ratio between the unique reads for the taxon and the number of observed unique reads of normalization molecules.

The amount of mcfDNA plasma concentration in each sample can then be quantified by using the measured relative abundance of the synthetic molecules initially spiked in the plasma.

In some cases, testing with plasma mcfDNA-seq is performed on available samples collected between seven days before and four days after each BSI episode, and two negative control samples are added for each BSI episode. In some cases, the samples are collected at least three days prior to a bloodstream infection of invasive fungal infection. The laboratory can be blinded to expected results until sequencing is completed and reported.

Analysis

Disclosed herein in some embodiments, are methods of analyzing nucleic acids. Such analytical methods include sequencing the nucleic acids as well as bioinformatic analysis of the sequencing results (e.g., sequence reads).

In some embodiments, a sequencing is performed using a next generation sequencing assay. As used herein, the term “next generation” generally refers to any high-throughput sequencing approach including, but not limited to one or more of the following: massively-parallel signature sequencing, pyrosequencing (e.g., using a Roche 454 Genome Analyzer™ sequencing device), Illumina™ (Solexa™) sequencing (e.g., using an Illumina NextSeq™ 500), sequencing by synthesis (Illumina™), ion semiconductor sequencing (Ion torrent™), sequencing by ligation (e.g., SOLiD™ sequencing), single molecule real-time (SMRT) sequencing (e.g., Pacific Bioscience™), polony sequencing, DNA nanoball sequencing (Complete Genomics™), heliscope single molecule sequencing (Helicos Biosciences™), metagenomic sequencing and nanopore sequencing (e.g., Oxford Nanopore™). In some embodiments, a sequencing assay can comprise nanopore sequencing. In some embodiments, a sequencing assay can include some form of Sanger sequencing. In some embodiments, a sequencing can involve shotgun sequencing; in some embodiments, a sequencing can include bridge amplification PCR.

In some embodiments, a sequencing assay comprises a Gilbert's sequencing method. In some embodiments, a Gilbert's sequencing method can comprise chemically modifying nucleic acids (e.g., DNA) and then cleaving them at specific bases. In some embodiments, a sequencing assay can comprise dideoxynucleotide chain termination or Sanger-sequencing.

In some embodiments, a sequencing-by-synthesis approach is used in the methods provided herein. In some embodiments, fluorescently-labeled reversible-terminator nucleotides are introduced to clonally-amplified DNA templates immobilized on the surface of a glass flowcell. During each sequencing cycle, a single labeled deoxynucleoside triphosphate (dNTP) may be added to the nucleic acid chain. The labeled terminator nucleotide may be imaged when added in order to identify the base and then the terminator group may be enzymatically cleaved to allow synthesis of the strand to proceed. A terminator group can comprise a 3′-O-blocked reversible terminator or a 3′-unblocked reversible terminator. Since all four reversible terminator-bound dNTPs (A, C, T, G) are generally present as single, separate molecules, natural competition may minimize incorporation bias.

In some embodiments, a method called Single-molecule real-time (SMRT) is used. In such approach, nucleic acids (e.g., DNA) are synthesized in zero-mode wave-guides (ZMWs), which are small well-like containers with capturing tools located at the bottom of the well. The sequencing is performed with use of unmodified polymerase (attached to the ZMW bottom) and fluorescently labelled nucleotides flowing freely in the solution. The fluorescent label is detached from the nucleotide upon its incorporation into the DNA strand, leaving an unmodified DNA strand. A detector such as a camera may then be used to detect the light emissions; and the data may be analyzed bioinformatically to obtain sequence information.

In some embodiments, a sequencing by ligation approach is used to sequence the nucleic acids in a sample. One example is the next generation sequencing method of SOLiD (Sequencing by Oligonucleotide Ligation and Detection) sequencing (Life Technologies). This next generation technology may generate hundreds of millions to billions of small sequence reads at one time. The sequencing method may comprise preparing a library of DNA fragments from the sample to be sequenced. In some embodiments, the library is used to prepare clonal bead populations in which only one species of fragment is present on the surface of each bead (e.g., magnetic bead). The fragments attached to the magnetic beads may have a universal P1 adapter sequence attached so that the starting sequence of every fragment is both known and identical. In some embodiments, the method may further involve PCR or emulsion PCR. For example, the emulsion PCR may involve the use of microreactors containing reagents for PCR. The resulting PCR products attached to the beads may then be covalently bound to a glass slide. A sequencing assay such as a SOLiD sequencing assay or other sequencing by ligation assay may include a step involving the use of primers. Primers may hybridize to the P1 adapter sequence or other sequence within the library template. The method may further involve introducing four fluorescently labelled di-base probes that compete for ligation to the sequencing primer. Specificity of the di-base probe may be achieved by interrogating every first and second base in each ligation reaction. Multiple cycles of ligation, detection and cleavage may be performed with the number of cycles determining the eventual read length. In some embodiments, following a series of ligation cycles, the extension product can be removed and the template can be reset with a primer complementary to the n-1 position for a second round of ligation cycles. Multiple rounds (e.g., 5 rounds) of primer reset may be completed for each sequence tag. Through the primer reset process, each base may be interrogated in two independent ligation reactions by two different primers. For example, a base at read position 5 can be assayed by primer number 2 in ligation cycle 2 and by primer number 3 in ligation cycle 1.

In some embodiments, a detection or quantification analysis of oligonucleotides can be accomplished by sequencing. In some embodiments, entire synthesized oligonucleotides can be detected via full sequencing of all oligonucleotides by e.g., Illumina HiSeq 2500™, including the sequencing methods described herein.

In some embodiments, the sequencing is accomplished through classic Sanger sequencing methods. Sequencing can also be accomplished using high-throughput systems some of which allow detection of a sequenced nucleotide immediately after or upon its incorporation into a growing strand, e.g., detection of sequence in real time or substantially real time. In some embodiments, high throughput sequencing generates at least 1,000, at least 5,000, at least 10,000, at least 20,000, at least 30,000, at least 40,000, at least 50,000, at least 100,000, or at least 500,000 sequence reads per hour. In some embodiments, each read is at least 50, at least 60, at least 70, at least 80, at least 90, at least 100, at least 120, or at least 150 bases per read. In some embodiments, each read is up to 2000, up to 1000, up to 900, up to 800, up to 700, up to 600, up to 500, up to 400, up to 300, up to 200, or up to 100 bases per read. Long read sequencing can include sequencing that provides a contiguous sequence read of longer than 500 bases, longer than 800 bases, longer than 1000 bases, longer than 1500 bases, longer than 2000 bases, longer than 3000 bases, or longer than 4500 bases per read.

In some embodiments, a high-throughput sequencing can involve the use of technology available by Illumina's Genome Analyzer IIX™, MiSeq personal sequencer™, or HiSeq™ systems, such as those using HiSeq 2500™, HiSeq 1500™, HiSeq 2000™, or HiSeq 1000™. These machines use reversible terminator-based sequencing by synthesis chemistry. These machines can sequence 200 billion or more reads in eight days. Smaller systems may be utilized for runs within 3, 2, or 1 days or less time. Short synthesis cycles may be used to minimize the time it takes to obtain sequencing results.

In some embodiments, a high-throughput sequencing involves the use of technology available by ABI Solid System. This genetic analysis platform can enable massively parallel sequencing of clonally-amplified DNA fragments linked to beads. The sequencing methodology is based on sequential ligation with dye-labeled oligonucleotides.

In some embodiments, a next-generation sequencing can comprise ion semiconductor sequencing (e.g., using technology from Life Technologies™ (Ion Torrent™)). Ion semiconductor sequencing can take advantage of the fact that when a nucleotide is incorporated into a strand of DNA, an ion can be released. To perform ion semiconductor sequencing, a high-density array of micromachined wells can be formed. Each well can hold a single DNA template. Beneath the well can be an ion sensitive layer, and beneath the ion sensitive layer can be an ion sensor. When a nucleotide is added to a DNA, an H⁺ ion can be released, which can be measured as a change in pH. The H⁺ ion can be converted to voltage and recorded by the semiconductor sensor. An array chip can be sequentially flooded with one nucleotide after another. In some embodiments, no scanning, light, or cameras are required. In some embodiments, an IONPROTON™ Sequencer is used to sequence nucleic acid. In some embodiments, an IONPGM™ Sequencer is used. The Ion Torrent Personal Genome Machine™ (PGM) can sequence 10 million reads in two hours.

In some embodiments, a high-throughput sequencing involves the use of technology available by Helicos BioSciences Corporation™ (Cambridge, Mass.) such as the Single Molecule Sequencing by Synthesis (SMSS) method. SMSS can allow for sequencing the entire human genome in up to 24 hours. In some embodiments, SMSS may not require a pre amplification step prior to hybridization. In some embodiments, SMSS may not require any amplification. In some embodiments, methods of using SMSS are described in part in US Publication Application Nos. 20060024711; 20060024678; 20060012793; 20060012784; and 20050100932, each of which are herein incorporated by reference.

In some embodiments, a high-throughput sequencing involves the use of technology available by 454 Lifesciences, Inc.™ (Branford, Conn.) such as the Pico Titer Plate™ device which includes a fiber optic plate that transmits chemiluminescent signal generated by the sequencing reaction to be recorded by a charge-coupled device (CCD) camera in the instrument. This use of fiber optics can allow for the detection of a minimum of 20 million base pairs in 4.5 hours. In some embodiments, methods for using bead amplification followed by fiber optics detection are described in Marguiles, M., et al. “Genome sequencing in microfabricated high-density picolitre reactors”, Nature, doi: 10.1038/nature03959; which is herein incorporated by reference.

In some embodiments, high-throughput sequencing is performed using Clonal Single Molecule Array (Solexa, Inc.™) or sequencing-by-synthesis (SBS) utilizing reversible terminator chemistry. Methods of using these technologies are described in part in U.S. Pat. Nos. 6,969,488; 6,897,023; 6,833,246; 6,787,308; and US Publication Application Nos. 20040106110; 20030064398; 20030022207; and Constans, A., The Scientist 2003, 17(13):36, each of which are herein incorporated by reference.

In some embodiments, the next generation sequencing is nanopore sequencing. A nanopore can be a small hole, e.g., on the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from Oxford Nanopore Technologies™; e.g., a GridION™ system. A single nanopore can be inserted in a polymer membrane across the top of a microwell. Each microwell can have an electrode for individual sensing. The microwells can be fabricated into an array chip, with 100,000 or more microwells (e.g., more than 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiNx, or SiO₂). The nanopore can be a hybrid pore (e.g., an integration of a protein pore into a solid-state membrane). The nanopore can be a nanopore with an integrated sensors (e.g., tunneling electrode detectors, capacitive detectors, or graphene based nano-gap or edge state detectors (see e.g., Garaj et al. (2010) Nature vol. 67, doi: 10.1038/nature09379)). A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some embodiments, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases.

In some embodiments, a nanopore sequencing technology from GENIA™ can be used. An engineered protein pore can be embedded in a lipid bilayer membrane. “Active Control” technology can be used to enable efficient nanopore-membrane assembly and control of DNA movement through the channel. In some embodiments, the nanopore sequencing technology is from NABsys™. Genomic DNA can be fragmented into strands of average length of about 100 kb. The 100 kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing By Hybridization (mwSBH).” In some embodiments, the nanopore sequencing technology is from IBM™ or Roche™. An electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

The next generation sequencing can comprise DNA nanoball sequencing (as performed, e.g., by Complete Genomics™; see e.g., Drmanac et al. (2010) Science 327: 78-81, which is incorporated herein by reference). DNA can be isolated, fragmented, and size selected. For example, DNA can be fragmented (e.g., by sonication) to a mean length of about 500 bp. Adaptors (Adl) can be attached to the ends of the fragments. The adaptors can be used to hybridize to anchors for sequencing reactions. DNA with adaptors bound to each end can be PCR amplified. The adaptor sequences can be modified so that complementary single strand ends bind to each other forming circular DNA. The DNA can be methylated to protect it from cleavage by a type IIS restriction enzyme used in a subsequent step. An adaptor (e.g., the right adaptor) can have a restriction recognition site, and the restriction recognition site can remain non-methylated. The non-methylated restriction recognition site in the adaptor can be recognized by a restriction enzyme (e.g., Acul), and the DNA can be cleaved by Acul 13 bp to the right of the right adaptor to form linear double stranded DNA. A second round of right and left adaptors (Ad2) can be ligated onto either end of the linear DNA, and all DNA with both adapters bound can be PCR amplified (e.g., by PCR). Ad2 sequences can be modified to allow them to bind each other and form circular DNA. The DNA can be methylated, but a restriction enzyme recognition site can remain non-methylated on the left Adl adapter. A restriction enzyme (e.g., Acul) can be applied, and the DNA can be cleaved 13 bp to the left of the Adl to form a linear DNA fragment. A third round of right and left adaptor (Ad3) can be ligated to the right and left flank of the linear DNA, and the resulting fragment can be PCR amplified. The adaptors can be modified so that they can bind to each other and form circular DNA. A type III restriction enzyme (e.g., EcoP15) can be added; EcoP15 can cleave the DNA 26 bp to the left of Ad3 and 26 bp to the right of Ad2. This cleavage can remove a large segment of DNA and linearize the DNA once again. A fourth round of right and left adaptors (Ad4) can be ligated to the DNA, the DNA can be amplified (e.g., by PCR), and modified so that they bind each other and form the completed circular DNA template.

Rolling circle replication (e.g., using Phi 29 DNA polymerase) can be used to amplify small fragments of DNA. The four adaptor sequences can contain palindromic sequences that can hybridize and a single strand can fold onto itself to form a DNA nanoball (DNB™) which can be approximately 200-300 nanometers in diameter on average. A DNA nanoball can be attached (e.g., by adsorption) to a microarray (sequencing flowcell). The flow cell can be a silicon wafer coated with silicon dioxide, titanium and hexamethyldisilazane (HMDS) and a photoresistant material. Sequencing can be performed by unchained sequencing by ligating fluorescent probes to the DNA. The color of the fluorescence of an interrogated position can be visualized by a high-resolution camera. The identity of nucleotide sequences between adaptor sequences can be determined.

The methods provided herein may include use of a system that contains a nucleic acid sequencer (e.g., DNA sequencer, RNA sequencer) for generating DNA or RNA sequence information. The system may include a computer comprising software that performs bioinformatic analysis on the DNA or RNA sequence information. Bioinformatic analysis can include, without limitation, assembling sequence data, detecting and quantifying genetic variants in a sample, including germline variants and somatic cell variants (e.g., a genetic variation associated with cancer or pre-cancerous condition, a genetic variation associated with infection, or a combination thereof).

Sequencing data may be used to determine genetic sequence information, ploidy states, the identity of one or more genetic variants, as well as a quantitative measures of the variants, including relative and absolute relative measures.

In some embodiments a sequencing can involve sequencing of a genome. In some embodiments a genome can be that of a microbe or pathogen as disclosed herein. In some embodiments, sequencing of a genome can involve whole genome sequencing or partial genome sequencing. In some embodiments, a sequencing can be unbiased and can involve sequencing all or substantially all (e.g., greater than 70%, 80%, 90%) of the nucleic acids in a sample. In some embodiments, a sequencing of a genome can be selective, e.g., directed to portions of a genome of interest. In some embodiments, sequencing of select genes, or portions of genes may suffice for a desired analysis. In some embodiments, polynucleotides mapping to specific loci in a genome can be isolated for sequencing by, for example, sequence capture or site-specific amplification.

In some embodiments disclosed herein, is a method comprising a process of analyzing, calculating, quantifying, or a combination thereof In some embodiments, a method can be used to determine quantities of bacterial and fungal sequence reads. In some embodiments, metrics can be generated to determine quantities of bacterial sequences, fungal sequences or a combination thereof.

In some embodiments, sensitivity of a test refers to a test's ability to correctly detect subjects with an infection who do actually have an infection. In some embodiments, a sensitivity is a detection rate of a disease or infection. In some embodiments, a sensitivity is the proportion of people who test positive for a disease among those who have the disease. In some embodiments, a sensitivity can be calculated using the following formula: Sensitivity=(number of true positives)/(number of true positives+number of false negatives) or Sensitivity=(number of true positives)/(total number of sick individuals in a population); or Sensitivity=probability of a positive test when the patient has the disease or infection. In some embodiments, predictive sensitivity is the proportion of episodes for which mcfDNA sequencing identified the same organism subsequently identified in blood culture. For example, predictive sensitivity can be determined by the following ratio (number of true positives correctly identifying the microbe that later appears by blood culture)/(number of true positives correctly identifying the microbe that later appears by blood culture+number of false negatives).

In some embodiments, a sensitivity can comprise predicting that the subject at risk for the bloodstream infection will experience a symptom of a bloodstream infection (or become blood culture positive for a particular microbe) based on the amount of the pathogen associated with the bloodstream infection, wherein the predicting has a predictive sensitivity of at least about 50%, about 55%, about 60%, about 65%, about 70%, about 75%, about 80%, about 85%, about 90%, about 95%, or about 100% for samples collected at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 25 days, at least 28 days, or at least 30 days, prior to onset of a symptom of a fungal bloodstream infection or invasive fungal infection or prior to a positive blood culture indicating the presence of the fungal microbe. In some cases, the sensitivity is for a range of days within any range between 1-30 days (e.g., 2-10, 3-10, 4-10, 5-8, 7-25 days) prior to onset of a symptom of a fungal bloodstream, invasive fungal infection, or prior to a positive blood culture indicating the presence of the fungal microbe.

In some embodiments, a sensitivity can comprise predicting that the subject at risk for the bloodstream infection will experience a symptom of a bloodstream infection (or become blood culture positive for a particular microbe) based on the amount of the pathogen associated with the bloodstream infection, wherein the predicting has a predictive sensitivity of at least about 50%, about 55%, about 60%, about 65%, about 70%, about 75%, about 80%, about 85%, about 90%, about 95%, or about 100% for samples collected at least 1 day, at least 2 days, at least 3 days, at least 4 days, at least 5 days, at least 6 days, at least 7 days, at least 8 days, at least 9 days, or at least 10 days, prior to onset of a symptom of a bacterial bloodstream or prior to a positive blood culture indicating the presence of the bacterial microbe. In some cases, the sensitivity is for a range of days within any range between 1-10 days (e.g., 2-10, 3-10, 4-10, 5-8, 7-8 days) prior to onset of a symptom of a bacterial bloodstream or prior to a positive blood culture indicating the presence of the bacterial microbe.

In some embodiments, a specificity can refer to a test's ability to correctly reject healthy subjects without an infection. In some embodiments, a specificity of a test can comprise a proportion of subjects who truly do not have an infection who test negative for the infection. In some embodiments, a specificity can be calculated using the following formula: Specificity=(number of true negatives)/(number of true negatives+number of false positives) or Specificity=(number of true negatives)/(total number of well individuals in a population); or Specificity=probability of a negative test when the patient is healthy or well (or, depending on the context, when the patient does not later develop a sign of infection). In some cases, specificity is the proportion of negative control samples for which no bacterial or fungal organisms were identified by mcfDNA sequencing. In some cases, specificity is determined for common BSI pathogens (e.g., genera making up 1% or more of central-line associated BSI in the Children's Hospital Association Childhood Cancer and Blood Disorders Network BSI database). An exemplary list of such pathogens is provided in Table 1.

In some embodiments, the quantity for each organism identified in a method provided herein is expressed in Molecules Per Microliter (MPM), the number of DNA sequencing reads from the reported organism present per microliter of plasma. In some cases, detection or prediction of infection (or prediction of onset of symptoms of infection) occurs when the MPM is greater than a threshold value. In some cases, such threshold value of MPM may be greater than 10, 15, 20, 30, 50, 100, 200, 300, 400, 500, 600, 700, 800, 900, 1000, 2000, 5000, 7000, 10000, 20000, 30000, or 40000. In some cases, the MPM threshold is determined for a particular organism. For example, in some embodiments, the MPM threshold for detection of Escherichia (e.g., E. coli) or for prediction symptom onset related to Escherichia (e.g., E. coli) infection is greater than 400, 500, 600, 616, 700, 800, 1000, 2000, 5000, 10304, 20000, 30000, 400000, or 40165, e.g., 616-40165. In some embodiments, the MPM threshold for detection of Escherichia (e.g., E. coli) or for prediction symptom onset related to Escherichia (e.g., E. coli) infection is greater than 400, 500, 600, 700, 1000, 2000, 5000, 20000, 30000, 400000, or. In some embodiments, the MPM threshold for detection of Staphylococcus (e.g., S. epidermidis) or for prediction of symptom onset related to Staphylococcus (e.g., S. epidermidis) infection may be greater than 80, 143, 400, 500, 600, 700, 800, 1000, 1108, 2000, 2125, 5000, 7890, 10000, 20000, 20515, 30000, or 400000, or any range within these values. In some embodiments, the MPM threshold for detection of Enterococcus (e.g., E. faecium, E. gallinarum) or for prediction of symptom onset related to Enterococcus (e.g., E. faecium, E. gallinarum) infection may be greater than 400, 500, 600, 700, 753, 800, 1000, 2000, 4760, 5000, 7903, 10000, 10207, 20000, 30000, 320740, 400000, 1000000, 1145864, 1476869 or any range within these values. In some cases, the MPM threshold for detection of Enterococcus (e.g., E. faecium, E. gallinarum) or for prediction of symptom onset related to Enterococcus (e.g., E. faecium, E. gallinarum) infection is greater than 4760, 10207, 320740, 1145864. In some cases, the MPM threshold for detection of Enterococcus (e.g., E. faecium) or for prediction of symptom onset related to Enterococcus (e.g., E. faecium) infection is between 4760 and 1145864. In some embodiments, the MPM threshold for detection of Rhodotorula (e.g., R. mucilaginosa) or for prediction of symptom onset related to Rhodotorula (e.g., R. mucilaginosa) infection may be greater than 10, 15, 17, 256, 400, 500, 600, 700, 800, 1000, 2000, 4760, 5000, 8000, 8015, 10000, 10207, 20000, 30000, 320740, 400000, 1000000, or 1145864, or any range within these values. In some embodiments, the MPM threshold for detection of Corynebacterium (e.g., C. jeikeium) or for prediction of symptom onset related to Corynebacterium (e.g., C. jeikeium) infection may be greater than 10, 20, 50, 90, 98, 100, 400, 500, 600, 700, 800, 1000, 2000, 4760, 5000, 8000, 8015, 10000, 10207, 20000, 30000, 320740, 400000, 1000000, or 1145864, or any range within these values. In some embodiments, the MPM threshold for detection of a fungal infection (e.g., Aspergillus spp., Aspergillus flavus/oryzae, Mucor spp., Mucor velutinosus, Penicillum decumbens, Histoplasma, Histoplasma capsulatum, Malassezia globosa) or for prediction of symptom onset related to such fungal infection may be greater than 1, 5, 10, 15, 20, 25, 30, 40, 50, 75, 100, 150, 200, 300, 400, 500, 600, 700, 753, 800, 1000, 2000, 4760, 5000, 7903, 10000, 10207, 20000, 30000, 320740, 400000, 1000000, 1145864, 1476869 or any range within these values. In some cases, the MPM threshold for any of the preceding infections is “about” (as defined herein) any of the preceding values.

In some embodiments, a specificity can comprise predicting that the subject at risk for the bloodstream infection will experience a symptom of a bloodstream infection based on the amount of the pathogen associated with the bloodstream infection, wherein the predicting has a predictive specificity of at least about 50%, about 55%, about 60%, about 65%, about 70%, about 75%, about 80%, about 85%, about 90%, about 95%, or about 100% for samples collected at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, or at least 30 days prior to onset of at least one symptom or sign of a fungal bloodstream infection or invasive fungal infection. In some embodiments, a specificity can comprise predicting that the subject at risk for the bloodstream infection will experience a symptom of a bloodstream infection based on the amount of the pathogen associated with the bloodstream infection, wherein the predicting has a predictive specificity of at least about 50%, about 55%, about 60%, about 65%, about 70%, about 75%, about 80%, about 85%, about 90%, about 95%, or about 100% for samples collected at least 1 day, at least 2 days, at least 3 days, at least 4 days, at least 5 days, at least 6 days, at least 7 days, at least 8 days, at least 9 days, or at least 10 days, prior to onset of at least one symptom or sign of a bacterial bloodstream infection.

In some cases, the infection or risk of infection is detected following calculation of abundance of a microbe. In some embodiments, calculation of abundance of a microbe comprises calculating abundance of a certain type of microbe (e.g., abundance of bacteria, abundance of fungi). In some cases, the calculation comprises calculating abundance of a family of microbes, a genus of microbes, or a species of microbes. In some cases, the calculation of abundance comprises calculating abundance of common bloodstream infection pathogens, such abundance of at least 1, 2, 3, 4, 5, 10, 15 or all of the microbes listed in Table 1.

Treating

In some embodiments, the non-limiting methods provided herein can comprise administering a treatment to a subject prior to onset of an infection (e.g., bloodstream infection (BSI), bacterial bloodstream infection, invasive fungal infection), and, in some embodiments, prior to onset of one or more symptoms of infection (e.g., fever, elevated heart rate, low blood pressure, hyperventilation). In some embodiments, the treatment is administered to a subject when the subject is blood culture negative for the organism that is the target of the treatment. In some embodiments, the infection is detected or predicted by a method provided herein when the subject is blood culture negative, but the treatment is administered when the subject is blood culture positive. In some embodiments, the infection is detected or predicted by a method provided herein when the subject is blood culture negative, and the treatment is administered when the subject is blood culture negative. In some embodiments, the treatment is a preemptive treatment that prevents an asymptomatic infection from progressing into a symptomatic infection. In some embodiments, the treatment is a prophylactic treatment that prevents the onset of infection. In some embodiments, the treatment treats or reduces symptoms of an infection.

Various non-limiting treatments provided herein can be provided. In some embodiments, the treatment is a broad-spectrum antimicrobial drug or an antimicrobial drug that targets a specific microbe or a specific class of microbes. In some embodiments, the treatment targets bacteria and/or fungi, particularly any of the microbial organisms identified herein in Tables 2-4, and Tables 6-8, or mentioned in the Examples section of this application. In some embodiments, the subject is treated with a combination of drugs (e.g., a combination of multiple antibiotics, multiple anti-fungal drugs, or both antibiotics and antifungal drugs). In some embodiments, the subject is treated with a combination of broad-spectrum antibiotics, a combination of broad- and narrow-spectrum antibiotics, a combination of narrow-spectrum antibiotics, a combination of broad-spectrum antifungals, a combination of broad and narrow-spectrum antifungals, or a combination of narrow-spectrum antifungals. In some embodiments, the subject is treated with a broad-spectrum antibiotic, a narrow-spectrum antibiotic, a broad-spectrum antifungal, a narrow-spectrum antifungal, or any combination thereof.

In some embodiments, the treatment is an antimicrobial. In some embodiments, an antimicrobial comprises a β-lactam, an aminoglycoside, a quinolone, an oxazolidinone, a sulfonamide, a macrolide, a tetracycline, an ansamycin, a streptogramin, a lipopeptide, used singly, or in any combination thereof as used herein and/or as recommended by a clinician. In some embodiments, the treatment is a broad-spectrum treatment. In some embodiments, the broad-spectrum treatment is a broad-spectrum antibiotic, a broad-spectrum anti-bacterial drug, a broad-spectrum antifungal, or any combination thereof. As used herein, the term “broad spectrum antibiotic” generally refers to a drug that acts on both gram negative and gram-positive bacteria, that acts on multiple types of gram-negative bacteria, and/or that acts on multiple types of gram-positive bacteria. In some embodiments, the broad-spectrum treatment acts on multiple types of fungal infections. In some embodiments, the broad-spectrum drug is a broad-spectrum non-limiting examples include β-lactam penicillin such as flucloxacillin, ampicillin (or amoxicillin). In some embodiments, the broad-spectrum drug is a β-lactam such as cephalosporin antibiotic (e.g., ceftriaxone, cefepime). The cephalosporin drug can be, in some embodiments, a first, second, third or fourth generation cephalosporin drug. In some embodiments, the broad-spectrum antibiotic is a quinolone drug (e.g., levofloxacin), a carbopenem-type antibiotic (e.g., meropenem), or a metronidazole.

In some embodiments, the broad-spectrum treatment is an antifungal drug. In some embodiments, the antifungal drug is, for example, a cefepime, a clotrimazole, a econazole, a miconazole, a terbinafine, a fluconazole, a ketoconazole, a nystatin, an amphotericin B, or any other known antifungal drugs and/or a combination thereof.

In some embodiments, the treatment is a narrow-spectrum antimicrobial drug. In some embodiments, the narrow-spectrum antimicrobial drug is a vancomycin, a glycopeptidic antibiotic active against gram-positive bacteria. In some embodiments, the narrow-spectrum drug can comprise various narrow-spectrum drugs, for example, a flucytosine. In some embodiments, the narrow-spectrum drug can comprise an oxazolidinone, for example, a linezolid, a posizolid, a radezolid, a penicillin VK, or any combination thereof.

In some embodiments, the antimicrobial drug is a pill, a gel, a tablet, a coated tablet, or any combination thereof and can be administered to the subject orally. In some embodiments, the treatment using an anti-fungal can be administered to the subject topically. In some embodiments, a topical administration can comprise administering the treatment as a cream, a gel, an ointment, a wash such as shampoo, a spray, or any combination thereof. In some embodiments, a treatment can be administered in the form of a capsule, a tablet, a liquid, an injectable, a pessary or any combination thereof.

In some embodiments, the antimicrobial drug is formulated as an infusion, and can be administered to the subject intravenously via a needle or catheter.

### EXAMPLES

### Example 1

Introduction

Serious infections, especially bloodstream infections (BSIs), are among the most important complications affecting patients receiving treatment for cancer. An incident of BSI-related sepsis can cause death, multiorgan failure, and neurocognitive damage. Although a predictive test that enables preemptive, pathogen-directed therapy could reduce BSI-related morbidity and mortality, no validated test is apparently available. Novel metagenomic microbiologic diagnostics, including plasma microbial cell-free DNA sequencing (mcfDNA-seq), show promise as diagnostic tests, but systematic evaluation for BSI prediction may still be needed. This prospective pilot study tested the novel hypothesis that mcfDNA-seq can identify a causative pathogen in the days before BSI develops. Bloodstream infection (BSI) is a common, life-threatening complication of treatment for cancer. Predicting BSI before onset of clinical symptoms would enable preemptive therapy. A study was performed to estimate sensitivity and specificity of plasma microbial cell-free DNA sequencing (mcfDNA-seq) for predicting BSI in patients at high risk of life-threatening infection. The question to be investigated was whether plasma microbial cell-free DNA sequencing (mcfDNA-seq) might be able to predict bloodstream infection (BSI) in immunocompromised patients days before the onset of attributable symptoms.

Methods

A prospective pilot cohort study of mcfDNA-seq for predicting BSI in pediatric patients (<25 years of age) with relapsed or refractory cancers at St Jude Children's Research Hospital, a specialist quaternary pediatric hematology-oncology referral center, Remnant clinical blood samples were collected during chemotherapy and hematopoietic cell transplantation. Samples collected during the 7 days before and at onset of BSI episodes, along with negative control samples from study participants, underwent: blinded testing using a mcfDNA-seq test in a Clinical Laboratory Improvement Amendments/College of American Pathologists-approved laboratory.

Study Design and Ethics

This study was approved by the St Jude Children's Research Hospital Institutional Review Board and followed the Strengthening the Reporting of Observational Studies in Epidemiology (STROBE) reporting guideline.

Participants

Participants were pediatric patients receiving treatment for relapsed or refractory cancer at St Jude Children's Research Hospital. Informed consent and assent for participation were obtained. Participation continued until death, loss to follow-up, transfer of care, resolution of gastrointestinal graft-vs-host disease, 30 days after hematopoietic cell transplantation, or participant request.

Detailed Inclusion and Enrolment Criteria

Participants were younger than 25 years, receiving treatment for relapsed or refractory malignancy at St. Jude, and expected to continue care for at least 7 days. Participants were identified by direct contact with primary clinicians and from patient acceptance notices, and were recruited by study staff. Written informed consent for study participation was obtained from the participant or their legal representative as appropriate, and assent was obtained from participants older than 7 years.

Detailed Off-Study Criteria

Study participation was completed upon participant request, loss to follow-up or transfer of care, death of the participant, or at thirty days after HCT, whichever occurred first. Participants with severe gastrointestinal graft-versus-host disease (GVHD) following HCT remained on study until severe GVHD resolved.

Clinical Data

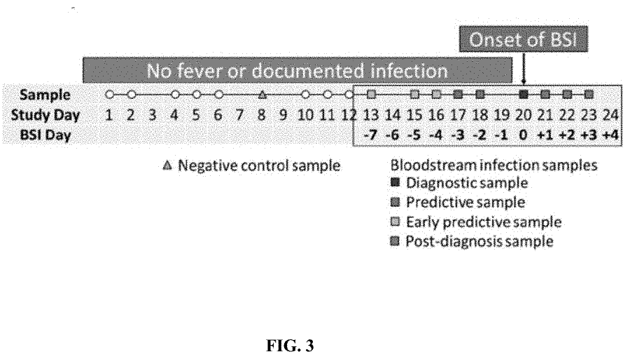

The Centers for Disease Control and Prevention's National Healthcare Safety Network definitions were used for BSI, with onset defined at the time of collection of the first positive blood culture. Institutional practice was to collect blood cultures in all episodes of fever or suspected infection. The a priori predictive period comprised the 3 days before BSI onset (FIG. 3). FIG. 3 shows the method of selection of plasma samples for testing as negative controls and bloodstream infection (BSI) episodes. Closed shapes represent available samples for an individual participant; negative control samples were defined as plasma samples obtained when no fever or infection was documented in the prior or subsequent 7 days; BSI samples were defined as plasma samples obtained in the 7 days before or 4 days after the onset of BSI. Samples obtained on the day of onset of BSI were classified as diagnostic samples; samples obtained on the 3 days before the onset of BSI were classified as predictive samples. Negative control samples were obtained from participants on a day for which no fever or infection was documented within the prior or subsequent 7 days. Participants could contribute multiple BSI and negative control episodes.

Clinical data were collected prospectively from the electronic medical record and from pharmacy and laboratory databases from 30 days before enrollment until 7 days after study completion. BSI episodes were defined according to National Healthcare Safety Network definitions for laboratory-confirmed bloodstream infection (CDC 2017). The onset of each BSI was defined as the date of collection of the first positive culture; institutional practice is to collect blood cultures from patients with a new onset fever or suspected systemic infection. The a priori-defined Predictive Period comprised the three days before BSI onset, and prediction during the period 4-7 days before BSI onset was evaluated as a secondary objective (FIG. 3). Negative control samples were obtained from participants on a day for which no fever or infection was documented within the prior or subsequent seven days. Participants could contribute multiple BSI episodes and negative control samples, but a minimum of 21 days was required between negative control samples.

Laboratory Procedures

Remnant blood collected in EDTA and available after the completion of clinical testing was stored at 4° C. until processed by centrifugation to plasma and stored at −80° C. until thawed for testing. Leftover blood was available from most samples collected for clinical hematology studies and was stored at 4° C. until processed to plasma and frozen.

Plasma mcfDNA-seq was performed in a Clinical Laboratory Improvement Amendments/College of American Pathologists—accredited laboratory. Briefly, cell-free DNA was extracted, DNA libraries were prepared, and sequencing was performed. Nonhuman sequencing reads were aligned to a curated pathogen database, and the concentration of pathogen-specific DNA fragments for each organism was reported in molecules per microliter (MPM). Available samples collected up to 7 days before each BSI episode were tested alongside 2 negative control samples per episode. The laboratory was blinded until results were finalized.

Plasma microbial cell-free DNA next generation sequencing (mcfDNA-seq) was performed using the clinical grade Karius Test, in a CLIA/CAP-accredited laboratory. Testing with plasma mcfDNA-seq was performed on available samples collected between 7 days before and 4 days after each BSI episode, and 2 negative control samples were added for each BSI episode. Samples were transported to Karius, Inc., overnight on dry ice and were confirmed to be frozen on arrival. BSI samples and negative control samples were sent in the same batch, and the laboratory was blinded to expected results until sequencing was completed and reported.

Reference Database and Quality Control

Reference genomes for Homo sapiens and microorganisms (bacteria, viruses, fungi/molds, and other eukaryotic pathogens) were retrieved from the National Center for Biotechnology Information (NCBI) ftp site (NCBI, U.S. National Library of Medicine (NLM), Human Genome: https://www.ncbi.nlm.nih.gov/genome/guide/human/, release GRCh38.p7), (NCBI, U.S. NLM, Microbial Genomes: https://www.ncbi.nlm.nih.gov/genome/microbes/). Sequence similarities between microorganism references were inspected to identify taxonomic mislabeling and sequence contamination. From the reference genomes passing these quality controls, a subset was selected to maximize sequence diversity. As part of the selection process, NCBI BioSample data (NCBI, U.S. NLM, Biosample: https://www.ncbi.nlm.nih.gov/biosample/), were used to ensure the inclusion of reference genomes from both clinical and non-clinical isolates. The final reference genome dataset included over 21,000 reference genomes, containing over 2.7 million sequences. Selected sequences were collected into a single FASTA file and used to generate the microorganism reference BLAST database.

A subset of these taxa, including 1251 clinically significant microorganisms, was used as the clinical reportable range, as follows:

Bacteria and Archaea

Methanobrevibacter smithii; Abiotrophia defectiva; Achromobacter denitrificans; Achromobacter insolitus; Achromobacter ruhlandii; Achromobacter xylosoxidans; Acidaminococcus intestini; Acidovorax citrulli; Acinetobacter baumannii; Acinetobacter bereziniae; Acinetobacter calcoaceticus; Acinetobacter haemolyticus; Acinetobacter nosocomialis; Acinetobacter pittii; Acinetobacter radioresistens; Acinetobacter seifertii; Acinetobacter soli; Acinetobacter ursingii; Actinobacillus ureae; Actinomadura latina; Actinomadura madurae; Actinomyces cardiffensis; Actinomyces europaeus; Actinomyces georgiae; Actinomyces gerencseriae; Actinomyces graevenitzii; Actinomyces israelii; Actinomyces massiliensis; Actinomyces meyeri; Actinomyces neuii; Actinomyces odontolyticus; Actinomyces oris; Actinomyces timonensis; Actinomyces turicensis; Actinomyces urogenitalis; Actinomyces viscosus; Aerococcus christensenii; Aerococcus sanguinicola; Aerococcus urinae; Aerococcus urinaehominis; Aerococcus viridans; Aeromonas caviae; Aeromonas enteropelogenes; Aeromonas hydrophila; Aeromonas schubertii; Aeromonas veronii; Aggregatibacter actinomycetemcomitans; Aggregatibacter aphrophilus; Aggregatibacter segnis; Agrobacterium tumefaciens; Alcaligenes faecalis; Alloiococcus otitis; Alloscardovia omnicolens; Anaerobiospirillum succiniciproducens; Anaerococcus hydrogenalis; Anaerococcus lactolyticus; Anaerococcus prevotii; Anaerococcus tetradius; Anaeroglobus geminatus; Anaplasma phagocytophilum; Arcanobacterium bernardiae; Arcanobacterium haemolyticum; Arcanobacterium pyogenes; Arcobacter butzleri; Arcobacter cryaerophilus; Arcobacter skirrowii; Atopobium parvulum; Atopobium rimae; Atopobium vaginae; Aureimonas altamirensis; Bacillus anthracis; Bacillus cereus; Bacillus circulans; Bacillus coagulans; Bacillus glycinifermentans; Bacillus licheniformis; Bacillus megaterium; Bacillus pumilus; Bacillus sphaericus; Bacillus subtilis; Bacillus thuringiensis; Bacteroides caccae; Bacteroides distasonis; Bacteroides eggerthii; Bacteroides forsythus; Bacteroides fragilis; Bacteroides merdae; Bacteroides ovatus; Bacteroides stercorin; Bacteroides thetaiotaomicron; Bacteroides uniformis; Bacteroides vulgatus; Bartonella alsatica; Bartonella bacilliformis; Bartonella birtlesii; Bartonella bovis; Bartonella clarridgeiae; Bartonella doshiae; Bartonella elizabethae; Bartonella grahamii; Bartonella henselae; Bartonella koehlerae; Bartonella quintana; Bartonella rattaustraliani; Bartonella rochalimae; Bartonella schoenbuchensis; Bartonella taylorii; Bartonella tribocorum; Bartonella vinsonii; Bergeyella zoohelcum; Bifidobacterium adolescentis; Bifidobacterium breve; Bifidobacterium dentium; Bifidobacterium longum; Bifidobacterium scardovii; Bordetella bronchialis; Bordetella bronchiseptica; Bordetella flabilis; Bordetella hinzii; Bordetella holmesii; Bordetella parapertussis; Bordetella pertussis; Bordetella petrii; Bordetella trematum; Borrelia burgdorferi; Borrelia crocidurae; Borrelia duttonii; Borrelia hermsii; Borrelia hispanica; Borrelia mayonii; Borrelia miyamotoi; Borrelia parkeri; Borrelia persica; Borrelia recurrentis; Borrelia turicatae; Borreliella afzelii; Borreliella garinii; Brevibacillus brevis; Brevibacillus laterosporus; Brevibacterium casei; Brevundimonas diminuta; Brevundimonas vesicularis; Brucella abortus; Brucella canis; Brucella melitensis; Brucella suis; Budvicia aquatica; Burkholderia ambifaria; Burkholderia anthina; Burkholderia cenocepacia; Burkholderia cepacia; Burkholderia contaminans; Burkholderia gladioli; Burkholderia glumae; Burkholderia mallei; Burkholderia multivorans; Burkholderia pseudomallei; Burkholderia pyrrocinia; Burkholderia stabilis; Campylobacter coli; Campylobacter concisus; Campylobacter corcagiensis; Campylobacter cuniculorum; Campylobacter curvus; Campylobacter fetus; Campylobacter gracilis; Campylobacter hominis; Campylobacter hyointestinalis; Campylobacter iguaniorum; Campylobacter jejuni; Campylobacter lari; Campylobacter mucosalis; Campylobacter showae; Campylobacter sputorum; Campylobacter upsaliensis; Campylobacter ureolyticus; Capnocytophaga canimorsus; Capnocytophaga cynodegmi; Capnocytophaga gingivalis; Capnocytophaga granulosa; Capnocytophaga haemolytica; Capnocytophaga ochracea; Capnocytophaga sputigena; Cardiobacterium hominis; Cardiobacterium valvarum; Catabacter hongkongensis; Cedecea neteri; Cellulomonas flavigena; Chlamydia psittaci<Chlamydophila psittaci>; Chlamydia trachomatis; Chlamydophila pneumoniae; Chromobacterium haemolyticum; Chromobacterium violaceum; Chryseobacterium gleum; Chryseobacterium indologenes; Citrobacter amalonaticus; Citrobacter braakii; Citrobacter farmeri; Citrobacter freundii; Citrobacter koseri; Clostridium baratii; Clostridium bifermentans; Clostridium butyricum; Clostridium clostridioforme; Clostridium difficile; Clostridium haemolyticum; Clostridium innocuum; Clostridium neonatale; Clostridium novyi; Clostridium paraputrificum; Clostridium perfringens; Clostridium sordellii; Clostridium tetani; Comamonas kerstersii; Comamonas terrigena; Comamonas testosteroni; Corynebacterium accolens; Corynebacterium afermentans; Corynebacterium amycolatum; Corynebacterium argentoratense; Corynebacterium aurimucosum; Corynebacterium diphtheriae; Corynebacterium falsenii; Corynebacterium freiburgense; Corynebacterium freneyi; Corynebacterium glucuronolyticum; Corynebacterium halotolerans; Corynebacterium jeikeium; Corynebacterium kroppenstedtii; Corynebacterium kutscheri; Corynebacterium lipophiloflavum; Corynebacterium lymphophilum; Corynebacterium massiliense; Corynebacterium matruchotii; Corynebacterium minutissimum; Corynebacterium propinquum; Corynebacterium pseudodiphtheriticum; Corynebacterium pseudotuberculosis; Corynebacterium renale; Corynebacterium riegelii; Corynebacterium simulans; Corynebacterium stations; Corynebacterium striatum; Corynebacterium timonense; Corynebacterium tuscaniense; Corynebacterium ulcerans; Corynebacterium urealyticum; Corynebacterium ureicelerivorans; Corynebacterium xerosis; Coxiella burnetii; Cupriavidus gilardii; Cupriavidus metallidurans; Delftia acidovorans; Dermabacter hominis; Dermacoccus nishinomiyaensis; Dolosigranulum pigrum; Dysgonomonas capnocytophagoides; Dysgonomonas gadei; Dysgonomonas hofstadii; Dysgonomonas mossii; Edwardsiella hoshinae; Edwardsiella tarda; Eggerthella lenta; Ehrlichia canis; Ehrlichia chaffeensis; Ehrlichia muris; Eikenella corrodens; Elizabethkingia anophelis; Elizabethkingia meningoseptica; Elizabethkingia miricola; Empedobacter brevis; Empedobacter falsenii; Enterobacter aerogenes; Enterobacter amnigenus; Enterobacter cloacae complex; Enterobacter sakazakii; Enterococcus asini; Enterococcus avium; Enterococcus casseliflavus; Enterococcus cecorum; Enterococcus columbae; Enterococcus dispar; Enterococcus durans; Enterococcus faecalis; Enterococcus faecium; Enterococcus gallinarum; Enterococcus gilvus; Enterococcus haemoperoxidus; Enterococcus hirae; Enterococcus italicus; Enterococcus malodoratus; Enterococcus mundtii; Enterococcus pallens; Enterococcus phoeniculicola; Enterococcus pseudoavium; Enterococcus raffinosus; Enterococcus saccharolyticus; Enterococcus sulfureus; Enterococcus thailandicus; Erysipelothrix rhusiopathiae; Escherichia albertii; Escherichia blattae; Escherichia coli; Escherichia fergusonii; Escherichia hermannii; Escherichia vulneris; Eubacterium limosum; Eubacterium nodatum; Facklamia hominis; Facklamia sourekii; Filifactor alocis; Finegoldia magna; Francisella hispaniensis; Francisella noatunensis; Francisella philomiragia; Francisella tularensis; Franconibacter helveticus; Fusobacterium mortiferum; Fusobacterium necrophorum; Fusobacterium nucleatum; Fusobacterium periodonticum; Fusobacterium ulcerans; Fusobacterium varium; Gardnerella vaginalis; Gemella bergeri; Gemella haemolysans; Gemella morbillorum; Gemella sanguinis; Gordonia bronchialis; Gordonia rubripertincta; Gordonia terrae; Gordonibacter pamelaeae; Granulibacter bethesdensis; Granulicatella adiacens; Granulicatella elegans; Grimontia hollisae; Haemophilus aegyptius; Haemophilus ducreyi; Haemophilus haemolyticus; Haemophilus influenzae; Haemophilus parahaemolyticus; Haemophilus parainfluenzae; Haemophilus paraphrohaemolyticus; Haemophilus quentini; Haemophilus sputorum; Hafnia alvei; Hafnia paralvei; Helicobacter bilis; Helicobacter cinaedi; Helicobacter fennelliae; Helicobacter magdeburgensis; Helicobacter pylori; Isoptericola variabilis; Janibacter indicus; Jonesia denitrificans; Kerstersia gyiorum; Kingella denitrificans; Kingella kingae; Kingella oralis; Klebsiella michiganensis; Klebsiella oxytoca; Klebsiella pneumoniae; Klebsiella quasipneumoniae; Klebsiella variicola; Kluyvera ascorbata; Kluyvera cryocrescens; Kluyvera intermedia; Kocuria kristinae; Kocuria rhizophila; Kytococcus sedentarius; Lactobacillus acidophilus; Lactobacillus casei; Lactobacillus crispatus; Lactobacillus fermentum; Lactobacillus gasseri; Lactobacillus iners; Lactobacillus jensenii; Lactobacillus plantarum; Lactobacillus rhamnosus; Lactobacillus sakei; Lactobacillus ultunensis; Lactococcus garvieae; Laribacter hongkongensis; Leclercia adecarboxylata; Legionella anisa; Legionella birminghamensis; Legionella bozemanae; Legionella brunensis; Legionella cherrii; Legionella cincinnatiensis; Legionella clemsonensis; Legionella drancourtii; Legionella dumoffii; Legionella fairfieldensis; Legionella fallonii; Legionella feeleii; Legionella geestiana; Legionella hackeliae; Legionella jamestowniensis; Legionella jordanis; Legionella lansingensis; Legionella longbeachae; Legionella maceachernii; Legionella massiliensis; Legionella micdadei; Legionella moravica; Legionella norrlandica; Legionella oakridgensis; Legionella parisiensis; Legionella pneumophila; Legionella quinlivanii; Legionella sainthelensi; Legionella shakespearei; Legionella steelei; Legionella tucsonensis; Legionella wadsworthii; Legionella waltersii; Leifsonia aquatica; Leminorella grimontii; Leptospira alexanderi; Leptospira alstonii; Leptospira biflexa; Leptospira borgpetersenii; Leptospira broomii; Leptospira fainei; Leptospira inadai; Leptospira interrogans; Leptospira kirschneri; Leptospira kmetyi; Leptospira licerasiae; Leptospira mayottensis; Leptospira meyeri; Leptospira noguchii; Leptospira santarosai; Leptospira terpstrae; Leptospira vanthielii; Leptospira weilii; Leptospira wolbachii; Leptospira wolffii; Leptospira yanagawae; Leptotrichia buccalis; Leptotrichia goodfellowii; Leptotrichia shahii; Leptotrichia wadei; Leuconostoc citreum; Leuconostoc lactis; Leuconostoc mesenteroides; Leuconostoc pseudomesenteroides; Listeria grayi; Listeria innocua; Listeria ivanovii; Listeria monocytogenes; Listeria seeligeri; Listeria welshimeri; Mannheimia haemolytica; Megasphaera micronuciformis; Microbacterium foliorum; Microbacterium maritypicum; Microbacterium oxydans; Microbacterium paraoxydans; Microbacterium testaceum; Micrococcus luteus; Micrococcus lylae; Mobiluncus curtisii; Mobiluncus mulieris; Moellerella wisconsensis; Mogibacterium timidum; Moraxella atlantae; Moraxella catarrhalis; Moraxella lacunata; Moraxella lincolnii; Moraxella nonliquefaciens; Moraxella phenylpyruvica; Morganella morganii; Morococcus cerebrosus; Mycobacterium abscessus; Mycobacterium arupense; Mycobacterium asiaticum; Mycobacterium avium complex (MAC); Mycobacterium brisbanense; Mycobacterium canariasense; Mycobacterium chelonae; Mycobacterium chimaera; Mycobacterium conceptionense; Mycobacterium cosmeticum; Mycobacterium diernhoferi; Mycobacterium elephantis; Mycobacterium flavescens; Mycobacterium fortuitum; Mycobacterium franklinii; Mycobacterium genavense; Mycobacterium goodii; Mycobacterium gordonae; Mycobacterium grossiae; Mycobacterium haemophilum; Mycobacterium heckeshornense; Mycobacterium heraklionense; Mycobacterium holsaticum; Mycobacterium immunogenum; Mycobacterium intermedium; Mycobacterium iranicum; Mycobacterium kansasii; Mycobacterium koreense; Mycobacterium kumamotonense; Mycobacterium kyorinense; Mycobacterium leprae; Mycobacterium lepromatosis; Mycobacterium llatzerense; Mycobacterium mageritense; Mycobacterium malmoense; Mycobacterium marinum; Mycobacterium mucogenicum; Mycobacterium nebraskense; Mycobacterium neoaurum; Mycobacterium novocastrense; Mycobacterium obuense; Mycobacterium paraffinicum; Mycobacterium parascrofulaceum; Mycobacterium peregrinum; Mycobacterium phlei; Mycobacterium saopaulense; Mycobacterium scrofulaceum; Mycobacterium septicum; Mycobacterium setense; Mycobacterium sherrisii; Mycobacterium shigaense; Mycobacterium shimoidei; Mycobacterium simiae; Mycobacterium smegmatis; Mycobacterium szulgai; Mycobacterium talmoniae; Mycobacterium thermoresistibile; Mycobacterium triplex; Mycobacterium tuberculosis complex; Mycobacterium tusciae; Mycobacterium vaccae; Mycobacterium wolinskyi; Mycobacterium xenopi; Mycoplasma capricolum; Mycoplasma fermentans; Mycoplasma genitalium; Mycoplasma hominis; Mycoplasma hyopneumoniae; Mycoplasma penetrans; Mycoplasma pneumoniae; Mycoplasma pulmonis; Myroides marinus; Myroides odoratimimus; Myroides odoratus; Neisseria animaloris; Neisseria bacilliformis; Neisseria cinerea; Neisseria elongata; Neisseria flavescens; Neisseria gonorrhoeae; Neisseria lactamica; Neisseria meningitidis; Neisseria mucosa; Neisseria polysaccharea; Neisseria sicca; Neisseria wadsworthii; Neisseria weaveri; Neorickettsia helminthoeca; Neorickettsia sennetsu; Nocardia abscessus; Nocardia acidovorans; Nocardia africana; Nocardia alba; Nocardia amamiensis; Nocardia anaemiae; Nocardia aobensis; Nocardia araoensis; Nocardia arizonensis; Nocardia arthritidis; Nocardia asiatica; Nocardia beijingensis; Nocardia brasiliensis; Nocardia brevicatena; Nocardia caishijiensis; Nocardia carnea; Nocardia cerradoensis; Nocardia concava; Nocardia coubleae; Nocardia crassostreae; Nocardia cummidelens; Nocardia cyriacigeorgica; Nocardia dassonvillei; Nocardia elegans; Nocardia exalbida; Nocardia farcinica; Nocardia flavorosea; Nocardia fusca; Nocardia gamkensis; Nocardia grenadensis; Nocardia harenae; Nocardia higoensis; Nocardia ignorata; Nocardia inohanensis; Nocardia jejuensis; Nocardia jiangxiensis; Nocardia kruczakiae; Nocardia lijiangensis; Nocardia mexicana; Nocardia mikamii; Nocardia miyunensis; Nocardia niigatensis; Nocardia niwae; Nocardia nova; Nocardia otitidiscaviarum; Nocardia paucivorans; Nocardia pneumoniae; Nocardia pseudobrasiliensis; Nocardia pseudovaccinii; Nocardia puris; Nocardia rhamnosiphila; Nocardia salmonicida; Nocardia seriolae; Nocardia shimofusensis; Nocardia sienata; Nocardia soli; Nocardia speluncae; Nocardia takedensis; Nocardia tenerifensis; Nocardia terpenica; Nocardia testacea; Nocardia thailandica; Nocardia transvalensis; Nocardia uniformis; Nocardia vaccinii; Nocardia vermiculata; Nocardia veterana; Nocardia vinacea; Nocardia violaceofusca; Nocardia xishanensis; Nocardia yamanashiensis; Ochrobactrum anthropi; Ochrobactrum intermedium; Ochrobactrum oryzae; Odoribacter splanchnicus; Oerskovia turbata; Oligella ureolytica; Oligella urethralis; Olsenella uli; Oribacterium sinus; Orientia tsutsugamushi; Paenalcaligenes hominis; Paenibacillus alvei; Pandoraea apista; Pantoea agglomerans; Pantoea ananatis; Parabacteroides goldsteinii; Paraburkholderia fungorum; Paracoccus sanguinis; Paracoccus yeei; Parvimonas micra; Pasteurella bettyae; Pasteurella multocida; Pasteurella pneumotropica; Pediococcus acidilactici; Pediococcus pentosaceus; Peptoniphilus coxii; Peptoniphilus duerdenii; Peptoniphilus harei; Peptoniphilus indolicus; Peptoniphilus lacrimalis; Peptoniphilus rhinitidis; Peptostreptococcus anaerobius; Peptostreptococcus stomatis; Photobacterium damselae; Photorhabdus asymbiotica; Photorhabdus luminescens; Plesiomonas shigelloides; Pluralibacter gergoviae; Porphyromonas asaccharolytica; Porphyromonas gingivalis; Prevotella bivia; Prevotella buccae; Prevotella buccalis; Prevotella corporis; Prevotella denticola; Prevotella disiens; Prevotella intermedia; Prevotella loescheii; Prevotella melaninogenica; Prevotella nigrescens; Prevotella oralis; Prevotella oris; Propionibacterium acidifaciens; Propionibacterium granulosum; Propionibacterium namnetense; Propionibacterium propionicum; Proteus mirabilis; Proteus vulgaris; Providencia alcalifaciens; Providencia rettgeri; Providencia stuartii; Pseudomonas aeruginosa; Pseudomonas alcaligenes; Pseudomonas fluorescens; Pseudomonas fulva; Pseudomonas luteola; Pseudomonas mendocina; Pseudomonas mosselii; Pseudomonas oryzihabitans; Pseudomonas protegens; Pseudomonas pseudoalcaligenes; Pseudomonas putida; Pseudoramibacter alactolyticus; Psychrobacter cryohalolentis; Rahnella aquatilis; Ralstonia insidiosa; Ralstonia mannitolilytica; Ralstonia pickettii; Raoultella ornithinolytica; Raoultella planticola; Rhizobium pusense; Rhodococcus equi; Rhodococcus erythropolis; Rhodococcus fascians; Rhodococcus rhodochrous; Rickettsia akari; Rickettsia amblyommatis; Rickettsia australis; Rickettsia canadensis; Rickettsia conorii; Rickettsia felis; Rickettsia helvetica; Rickettsia honei; Rickettsia japonica; Rickettsia massiliae; Rickettsia monacensis; Rickettsia parkeri; Rickettsia prowazekii; Rickettsia raoultii; Rickettsia rickettsii; Rickettsia sibirica; Rickettsia slovaca; Rickettsia typhi; Riemerella anatipestifer; Roseobacter denitrificans; Roseomonas cervicalis; Roseomonas fauriae; Roseomonas gilardii; Roseomonas mucosa; Rothia aeria; Rothia dentocariosa; Rothia mucilaginosa; Salmonella bongori; Salmonella enterica; Sanguibacteroides justesenii; Serratia ficaria; Serratia fonticola; Serratia liquefaciens; Serratia marcescens; Serratia odorifera; Serratia plymuthica; Serratia rubidaea; Shewanella algae; Shewanella putrefaciens; Shigella boydii; Shigella dysenteriae; Shigella flexneri; Shigella sonnei; Siccibacter turicensis; Slackia exigua; Sneathia sanguinegens; Solobacterium moorei; Sphingobacterium spiritivorum; Staphylococcus agnetis; Staphylococcus argenteus; Staphylococcus arlettae; Staphylococcus aureus; Staphylococcus auricularis; Staphylococcus capitis; Staphylococcus caprae; Staphylococcus carnosus; Staphylococcus caseolyticus; Staphylococcus chromogenes; Staphylococcus cohnii; Staphylococcus condimenti; Staphylococcus epidermidis; Staphylococcus equorum; Staphylococcus gallinarum; Staphylococcus haemolyticus; Staphylococcus hominis; Staphylococcus hyicus; Staphylococcus lentus; Staphylococcus lugdunensis; Staphylococcus pasteuri; Staphylococcus pettenkoferi; Staphylococcus pseudintermedius; Staphylococcus saprophyticus; Staphylococcus schleiferi; Staphylococcus sciuri; Staphylococcus simiae; Staphylococcus simulans; Staphylococcus succinus; Staphylococcus vitulinus; Staphylococcus warneri; Staphylococcus xylosus; Stenotrophomonas acidaminiphila; Stenotrophomonas maltophilia; Streptobacillus moniliformis; Streptococcus agalactiae; Streptococcus anginosus; Streptococcus canis; Streptococcus constellatus; Streptococcus cricetus; Streptococcus cristatus; Streptococcus dentisani; Streptococcus dysgalactiae; Streptococcus equi; Streptococcus equinus; Streptococcus ferns; Streptococcus gallolyticus; Streptococcus gordonii; Streptococcus hyovaginalis; Streptococcus infantarius; Streptococcus infantis; Streptococcus iniae; Streptococcus intermedius; Streptococcus lutetiensis; Streptococcus macacae; Streptococcus macedonicus; Streptococcus massiliensis; Streptococcus mitis; Streptococcus mutans; Streptococcus oralis; Streptococcus parasanguinis; Streptococcus pasteurianus; Streptococcus peroris; Streptococcus pneumoniae; Streptococcus porcinus; Streptococcus pseudopneumoniae; Streptococcus pyogenes; Streptococcus ratti; Streptococcus salivarius; Streptococcus sanguinis; Streptococcus sobrinus; Streptococcus suis; Streptococcus thermophilus; Streptococcus tigurinus; Streptococcus uberis; Streptococcus vestibularis; Streptomyces cattleya; Streptomyces somaliensis; Sutterella wadsworthensis; Tatumella ptyseos; Terrisporobacter othiniensis; Treponema pallidum; Tropheryma whipplei; Tsukamurella paurometabola; Turicella otitidis; Ureaplasma parvum; Ureaplasma urealyticum; Veillonella dispar; Veillonella montpellierensis; Veillonella parvula; Vibrio albensis; Vibrio alginolyticus; Vibrio cholerae; Vibrio fluvialis; Vibrio furnissii; Vibrio harveyi; Vibrio metschnikovii; Vibrio mimicus; Vibrio navarrensis; Vibrio parahaemolyticus; Vibrio vulnificus; Weeksella virosa; Weissella confusa; Weissella paramesenteroides; Wohlfahrtiimonas chitiniclastica; Wolbachia pipientis; Xanthomonas axonopodis; Xylanimonas cellulosilytica; Yersinia enterocolitica; Yersinia frederiksenii; Yersinia intermedia; Yersinia kristensenii; Yersinia pestis; Yersinia pseudotuberculosis; Yersinia ruckeri; Yokenella regensburgei.

Fungi and Molds

Absidia glauca; Absidia repens; Acremonium chrysogenum; Acremonium furcatum; Actinomucor elegans; Alternaria alternata; Alternaria arborescens; Alternaria brassicicola; Anncaliia algerae; Apophysomyces elegans; Apophysomyces trapeziformis; Apophysomyces variabilis; Aspergillus aculeatus; Aspergillus arachidicola; Aspergillus bombycis; Aspergillus brasiliensis; Aspergillus calidoustus; Aspergillus campestris; Aspergillus candidus; Aspergillus carbonarius; Aspergillus chevalieri; Aspergillus clavatus; Aspergillus cristatus; Aspergillus fischeri; Aspergillus flavus; Aspergillus fumigatus; Aspergillus glaucus; Aspergillus hancockii; Aspergillus lentulus; Aspergillus luchuensis; Aspergillus nidulans; Aspergillus niger; Aspergillus nomius; Aspergillus novofumigatus; Aspergillus ochraceoroseus; Aspergillus oryzae; Aspergillus parasiticus; Aspergillus persii; Aspergillus rambellii; Aspergillus ruber; Aspergillus sclerotiorum; Aspergillus sojae; Aspergillus steynii; Aspergillus sydowii; Aspergillus taichungensis; Aspergillus terreus; Aspergillus thermomutatus; Aspergillus tubingensis; Aspergillus turcosus; Aspergillus udagawae; Aspergillus ustus; Aspergillus versicolor; Aspergillus wentii; Aspergillus westerdijkiae; Aureobasidium melanogenum; Aureobasidium namibiae; Aureobasidium pullulans; Aureobasidium subglaciale; Basidiobolus meristosporus; Beauveria bassiana; Beauveria rudraprayagi; Bipolaris papendorfii; Blastomyces dermatitidis; Blastomyces percursus; Byssochlamys spectabilis; Candida aaseri; Candida albicans; Candida apicola; Candida arabinofermentans; Candida auris; Candida boidinii; Candida bracarensis; Candida carpophila; Candida castellii; Candida dubliniensis; Candida duobushaemulonii; Candida ethanolica; Candida famata; Candida glabrata; Candida haemulonis; Candida homilentoma; Candida intermedia; Candida ipomoeae; Candida kefyr; Candida kipukae; Candida krusei; Candida lusitaniae; Candida nivariensis; Candida orthopsilosis; Candida parapsilosis; Candida pseudohaemulonii; Candida psychrophila; Candida sojae; Candida sorbophila; Candida sorboxylosa; Candida succiphila; Candida tanzawaensis; Candida tenuis; Candida tropicalis; Candida utilis; Candida versatilis; Capronia semi-immersa; Ceratocystis adiposa; Ceratocystis albifundus; Ceratocystis eucalypticola; Ceratocystis fimbriata; Ceratocystis manginecans; Ceratocystis platani; Cercospora fijiensis; Chaetomium globosum; Chaetomium thermophilum; Chrysosporium queenslandicum; Cladophialophora bantiana; Cladophialophora carrionii; Cladophialophora immunda; Cladophialophora psammophila; Cladophialophora yegresii; Cladosporium cladosporioides; Coccidioides immitis; Coccidioides posadasii; Cokeromyces recurvatus; Colletotrichum acutatum; Colletotrichum falcatum; Colletotrichum fioriniae; Colletotrichum gloeosporioides; Colletotrichum godetiae; Colletotrichum graminicola; Colletotrichum higginsianum; Colletotrichum incanum; Colletotrichum nymphaeae; Colletotrichum orbiculare; Colletotrichum salicis; Colletotrichum simmondsii; Colletotrichum sublineola; Colletotrichum tofieldiae; Conidiobolus coronatus; Conidiobolus incongruus; Coniosporium apollinis; Corynespora cassiicola; Cryptococcus bacillisporus; Cryptococcus bestiolae; Cryptococcus dejecticola; Cryptococcus deuterogattii; Cryptococcus fagi; Cryptococcus gattii; Cryptococcus neoformans; Cryptococcus pinus; Cryptococcus skinneri; Cryptococcus tetragattii; Cunninghamella; Curvularia lunata; Cyphellophora europaea; Debaryomyces fabryi; Diaporthe ampelina; Diaporthe aspalathi; Diaporthe longicolla; Emmonsia crescens; Emmonsia parva; Encephalitozoon cuniculi; Encephalitozoon hellem; Encephalitozoon intestinalis; Encephalitozoon romaleae; Enterocytozoon bieneusi; Exophiala alcalophila; Exophiala aquamarina; Exophiala calicioides; Exophiala dermatitidis; Exophiala mesophila; Exophiala oligosperma; Exophiala sideris; Exophiala spinifera; Exophiala xenobiotica; Filobasidium wieringae; Fonsecaea erecta; Fonsecaea monophora; Fonsecaea multimorphosa; Fonsecaea nubica; Fonsecaea pedrosoi; Fusarium agapanthi; Fusarium asiaticum; Fusarium avenaceum; Fusarium circinatum; Fusarium culmorum; Fusarium euwallaceae; Fusarium fujikuroi; Fusarium graminearum; Fusarium hostae; Fusarium langsethiae; Fusarium mangiferae; Fusarium meridionale; Fusarium nygamai; Fusarium oxysporum; Fusarium pininemorale; Fusarium poae; Fusarium praegraminearum; Fusarium proliferatum; Fusarium pseudograminearum; Fusarium sambucinum; Fusarium solani; Fusarium temperatum; Fusarium udum; Fusarium verticillioides; Geotrichum candidum; Hanseniaspora uvarum; Hansenula fabianii; Histoplasma capsulatum; Kluyveromyces lactis; Lachancea kluyveri; Lachancea lanzarotensis; Lachancea thermotolerans; Lachancea waltii; Leptosphaeria maculans; Lichtheimia corymbifera; Lichtheimia ramosa; Lodderomyces elongisporus; Lomentospora prolificans; Macrophomina phaseolina; Madurella mycetomatis; Malassezia caprae; Malassezia cuniculi; Malassezia dermatis; Malassezia equina; Malassezia furfur; Malassezia globosa; Malassezia japonica; Malassezia nana; Malassezia obtusa; Malassezia pachydermatis; Malassezia slooffiae; Malassezia sympodialis; Malassezia yamatoensis; Memnoniella echinata; Metarhizium acridum; Metarhizium album; Metarhizium anisopliae; Metarhizium brunneum; Metarhizium guizhouense; Metarhizium majus; Metarhizium rileyi; Metarhizium robertsii; Metschnikowia bicuspidata; Metschnikowia fructicola; Microsporum canis; Microsporum gypseum; Mortierella alpina; Mortierella elongata; Mortierella verticillata; Mucor ambiguus; Mucor circinelloides; Mucor indicus; Mucor irregularis; Mucor velutinosus; Myceliophthora thermophila; Nakaseomyces bacillisporus; Nakaseomyces delphensis; Nakazawaea peltata; Naumovozyma dairenensis; Nectria haematococca; Neofusicoccum parvum; Nosema apis; Nosema bombycis; Nosema ceranae; Ochroconis constricta; Ochroconis gallopava; Ogataea methanolica; Ogataea parapolymorpha; Ogataea polymorpha; Ophiostoma novo-ulmi; Ophiostoma piceae; Paecilomyces hepiali; Paracoccidioides brasiliensis; Paracoccidioides lutzii; Penicillium antarcticum; Penicillium brasilianum; Penicillium capsulatum; Penicillium carneum; Penicillium coprophilum; Penicillium decumbens; Penicillium digitatum; Penicillium expansum; Penicillium flavigenum; Penicillium freii; Penicillium griseofulvum; Penicillium islandicum; Penicillium italicum; Penicillium janthinellum; Penicillium marneffei; Penicillium nalgiovense; Penicillium nordicum; Penicillium occitanis; Penicillium oxalicum; Penicillium paneum; Penicillium paxilli; Penicillium piceum; Penicillium pinophilum; Penicillium purpurogenum; Penicillium roqueforti; Penicillium rubens; Penicillium sclerotiorum; Penicillium steckii; Penicillium subrubescens; Penicillium verruculosum; Penicillium vulpinum; Phaeoacremonium minimum; Phanerochaete carnosa; Phanerochaete chrysosporium; Phellinus noxius; Phialophora attae; Phoma herbarum; Phycomyces blakesleeanus; Pichia anomala; Pneumocystis carinii; Pneumocystis jirovecii; Pneumocystis murina; Pseudozyma hubeiensis; Purpureocillium lilacinum; Pyrenochaeta lycopersici; Pyrenochaeta mackinnonii; Ramichloridium mackenziei; Rasamsonia emersonii; Rhizoctonia solani; Rhizomucor miehei; Rhizomucor pusillus; Rhizomucor variabilis; Rhizopus delemar; Rhizopus microsporus; Rhizopus oryzae; Rhizopus stolonifer; Rhodotorula graminis; Rhodotorula mucilaginosa; Rhodotorula toruloides; Rhytidhysteron rufulum; Saccharomyces cerevisiae; Saksenaea oblongispora; Saksenaea vasiformis; Scedosporium apiospermum; Scedosporium aurantiacum; Scedosporium boydii; Scedosporium dehoogii; Schizophyllum commune; Sporopachydermia quercuum; Sporothrix brasiliensis; Sporothrix globosa; Sporothrix insectorum; Sporothrix pallida; Sporothrix schenckii; Stachybotrys chartarum; Stachybotrys chlorohalonata; Stemphylium lycopersici; Syncephalastrum monosporum; Syncephalastrum racemosum; Talaromyces amestolkiae; Talaromyces atroroseus; Talaromyces cellulolyticus; Talaromyces leycettanus; Talaromyces stipitatus; Talaromyces wortmannii; Thermoascus crustaceus; Thermomyces lanuginosus; Thielavia terrestris; Torulaspora delbrueckii; Trachipleistophora hominis; Trichoderma asperellum; Trichoderma atroviride; Trichoderma gamsii; Trichoderma hamatum; Trichoderma harzianum; Trichoderma longibrachiatum; Trichoderma parareesei; Trichoderma reesei; Trichoderma virens; Trichophyton; Trichophyton benhamiae; Trichosporon asahii; Trichosporon coremiiforme; Trichosporon cutaneum; Trichosporon faecale; Trichosporon guehoae; Trichosporon inkin; Trichosporon oleaginosus; Trichosporon ovoides; Trichosporon porosum; Ustilago cynodontis; Ustilago esculenta; Ustilago hordei; Ustilago maydis; Ustilago trichophora; Valsa mali; Verticillium alfalfae; Verticillium dahliae; Verticillium longisporum; Verticillium tricorpus; Vittaforma corneae; Volvariella volvacea; Wallemia ichthyophaga; Wallemia mellicola; Wickerhamomyces ciferrii; Yarrowia deformans; Yarrowia keelungensis; Yarrowia lipolytica

Other Eukaryota

Acanthamoeba; Ancylostoma ceylanicum; Ancylostoma duodenale; Angiostrongylus cantonensis; Angiostrongylus costaricensis; Anisakis simplex; Ascaris; Babesia divergens; Babesia microti; Balamuthia mandrillaris; Blastocystis hominis; Brugia malayi; Clonorchis sinensis; Cryptosporidium hominis; Cryptosporidium muris; Cryptosporidium parvum; Cryptosporidium ubiquitum; Cyclospora cayetanensis; Dirofilaria immitis; Dracunculus medinensis; Echinococcus granulosus; Echinococcus multilocularis; Echinostoma caproni; Entamoeba histolytica; Enterobius vermicularis; Fasciola hepatica; Giardia lamblia; Hymenolepis nana; Leishmania aethiopica; Leishmania amazonensis; Leishmania braziliensis; Leishmania donovani; Leishmania infantum; Leishmania major; Leishmania mexicana; Leishmania panamensis; Leishmania tropica; Loa loa; Naegleria fowleri; Necator americanus; Onchocerca volvulus; Opisthorchis viverrini; Plasmodium cynomolgi; Plasmodium falciparum; Plasmodium knowlesi; Plasmodium ovale; Plasmodium vivax; Pythium insidiosum; Schistosoma haematobium; Schistosoma japonicum; Schistosoma mansoni; Strongyloides stercoralis; Taenia asiatica; Taenia saginata; Taenia solium; Toxocara canis; Toxoplasma gondii; Trichinella; Trichomonas vaginalis; Trichuris trichiura; Trypanosoma brucei; Trypanosoma cruzi; Wuchereria bancrofti

Viruses

Adeno-associated dependoparvovirus A; Adeno-associated dependoparvovirus B; Alphapapillomavirus 1; Alphapapillomavirus 10; Alphapapillomavirus 11; Alphapapillomavirus 14; Alphapapillomavirus 2; Alphapapillomavirus 3; Alphapapillomavirus 4; Alphapapillomavirus 5; Alphapapillomavirus 6; Alphapapillomavirus 7; Alphapapillomavirus 8; Alphapapillomavirus 9; Betapapillomavirus 1; Betapapillomavirus 2; Betapapillomavirus 3; Betapapillomavirus 4; Betapapillomavirus 5; BK polyomavirus; Cowpox virus; Cytomegalovirus (CMV); Epstein-Barr virus (EBV); Gammapapillomavirus 1; Gammapapillomavirus 10; Gammapapillomavirus 11; Gammapapillomavirus 13; Gammapapillomavirus 14; Gammapapillomavirus 15; Gammapapillomavirus 16; Gammapapillomavirus 17; Gammapapillomavirus 19; Gammapapillomavirus 2; Gammapapillomavirus 3; Gammapapillomavirus 4; Gammapapillomavirus 5; Gammapapillomavirus 6; Gammapapillomavirus 7; Gammapapillomavirus 8; Gammapapillomavirus 9; Herpes B virus; Herpes simplex virus type 1 (HSV-1); Herpes simplex virus type 2 (HSV-2); Human adenovirus A; Human adenovirus B; Human adenovirus C; Human adenovirus D; Human adenovirus E; Human adenovirus F; Human bocavirus; Human herpesvirus 6A; Human herpesvirus 6B; Human herpesvirus 7; Human papillomavirus; Human papillomavirus 132-like viruses; Human papillomavirus type 136; Human papillomavirus type 140; Human papillomavirus type 154; Human papillomavirus type 167; Human parvovirus; Human polyomavirus 6; Human polyomavirus 7; JC polyomavirus; Kaposi sarcoma-associated herpesvirus; KI polyomavirus; Merkel cell polyomavirus; Molluscum contagiosum virus; Monkeypox virus; Mupapillomavirus 1; Mupapillomavirus 2; Nupapillomavirus 1; Orf virus; Porcine circovirus 1; Porcine circovirus 2; Primate bocaparvovirus 1; Pseudocowpox virus; STL polyomavirus; Tanapox virus; Torque teno virus; Torque teno virus 1; Torque teno virus 10; Torque teno virus 12; Torque teno virus 14; Torque teno virus 15; Torque teno virus 16; Torque teno virus 19; Torque teno virus 2; Torque teno virus 25; Torque teno virus 26; Torque teno virus 27; Torque teno virus 28; Torque teno virus 3; Torque teno virus 4; Torque teno virus 6; Torque teno virus 7; Torque teno virus 8; Trichodysplasia spinulosa-associated polyomavirus; Vaccinia virus; Varicella-zoster virus (VZV); Variola virus; WU Polyomavirus; Yaba monkey tumor virus.

Clinical Reportable Range (CRR)

The selection of organisms in the clinical reportable range (CRR) was performed as follows. A candidate list was generated by two board-certified Infectious Disease physicians by including (a) DNA viruses, culturable bacteria, additional fastidious and unculturable bacteria, mycobacteria, and eukaryotic pathogens from the standard text (CDC 2017) and a number of infectious disease references, (b) organisms in the pathogen database referenced in published case reports and (c) reference genomes sequenced from human clinical isolates (as indicated by NCBI's BioSample resource) with publications supporting pathogenicity. Organisms from the above list that were associated with high quality reference genomes, as determined by the reference database QC process (see above), were used to further narrow the range. Finally, organisms at risk of generating common false-positive calls because of sporadic environmental contamination were removed. The sequence database is continuously curated to minimize human cross-reactivity as well as cross-reactivity between pathogens and is screened to mitigate contamination with sequences from human or other organisms.

Sequencing

Plasma samples were thawed, centrifuged at 16,000 rcf for ten minutes, and spiked with a known concentration of synthetic DNA molecules for quality control purposes. Cell-free DNA was extracted from 0.5 mL plasma using a magnetic bead-based method (Omega Biotek, Norcross, Ga.). DNA libraries for sequencing are constructed using a modified Ovation® Ultralow System V2 library preparation kit (NuGEN, San Carlos, Calif.). Negative controls (buffer only instead of plasma) and positive controls (healthy plasma spiked with a known mixture of microbial DNA fragments) were processed alongside patient samples in every batch. Samples were multiplexed with other samples and sequenced on an Illumina NextSeq® 500.

Analysis Pipeline

Primary sequencing output files were processed using bcl2fastq (v2.17.1.14) to generate the demultiplexed sequencing reads files. Reads were filtered based on sequencing quality and trimmed based on partial or full adapter sequence. Bowtie2 (version 2.2.4) was used to align the remaining reads against the human and synthetic-molecules references. Sequencing reads exhibiting strong alignment against the human references or the synthetic molecule references were collected and excluded from further analysis. Remaining reads were aligned against a microorganism reference database using NCBI-blast (version 2.2.30+). A mixture model was used to assign a likelihood to the complete collection of sequencing reads that included the read sequence probabilities and the (unknown) abundances of each taxon in the sample. An expectation-maximization algorithm was applied to compute the maximum likelihood estimate of each taxon abundance. Only taxa whose abundances rejected the null hypothesis of originating from environmental contamination (as calculated from the negative controls) were reported. The quantity for each organism identified was expressed in Molecules Per Microliter (MPM), the number of DNA sequencing reads from the reported organism present per microliter of plasma. The entire process from DNA extraction through analysis was typically completed within 28 hours.

Power and Statistical Considerations

Simon's 2-stage design was applied for the primary objective. The minimal acceptable sensitivity was set at 30% and favorable sensitivity at 50% (Simon 1989). A favorable sensitivity of 50% was chosen as the approximate efficacy of antibacterial prophylaxis in this population (Gafter-Gvili 2012). An interim analysis was planned after accrual of predictive samples for 15 BSI episodes.

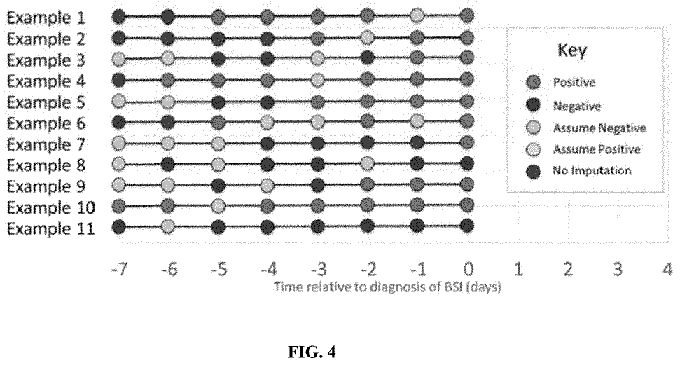

For the estimation of sensitivity and specificity, quantitative results were dichotomized as present or absent. The predictive sensitivity was estimated for the three-day period before BSI as a unit and separately for each of the seven days before BSI. The diagnostic sensitivity was estimated from the results of samples collected on the day of BSI onset. Predictive sensitivity was defined as the proportion of episodes for which mcfDNA-seq identified the same organism that was subsequently identified in blood culture. For polymicrobial BSI episodes, the identification by mcfDNA-seq of all organisms grown in culture was required to classify the organism present. The Wilson method was used to calculate 95% confidence intervals for proportions. Gaur 2017. For the estimation of sensitivity for each day before BSI onset, logical derivation was used to impute missing values by imputing that missing values between two positive results before day of diagnosis were positive, and negative results preceding negative results were negative, imputation was not used in other circumstances (FIG. 4). FIG. 4 shows the method of logical derivation for imputation of missing mcfDNA-seq results.

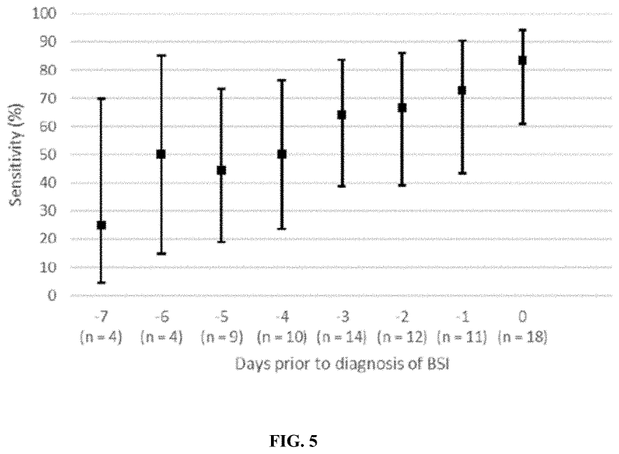

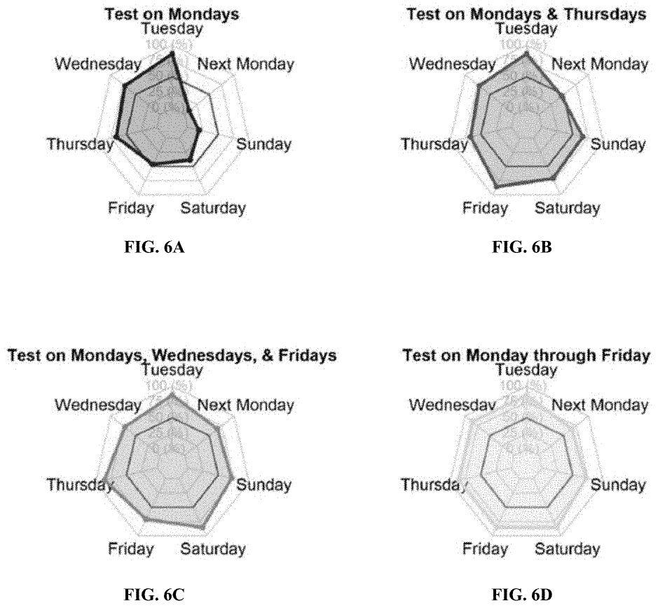

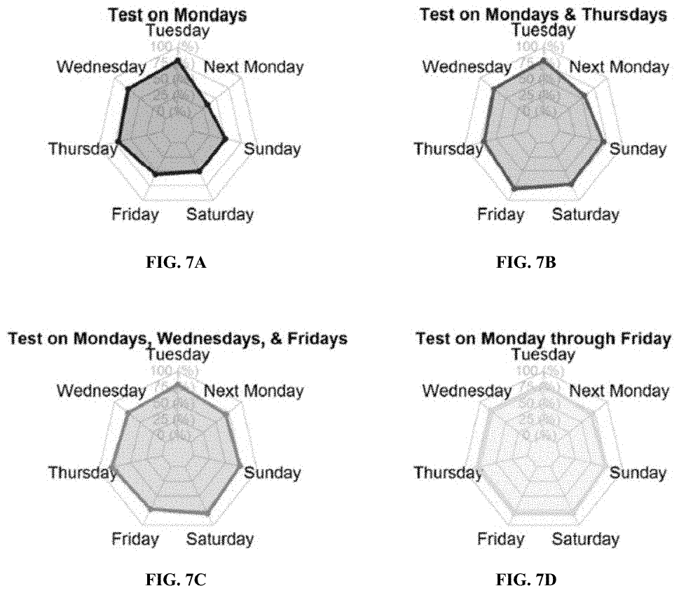

To evaluate the effect on sensitivity of different models of frequency of testing for bacterial BSI (FIG. 6A-6D and FIG. 7A-7D), estimated sensitivities were calculated for each day of the week, and minimum and median sensitivities were reported for each model. This analysis assumed that the risk of BSI events is identical for each day and that mcfDNA-seq test results would be available on the day of collection. The models evaluated were as follows: once-weekly testing on Mondays; twice-weekly testing on Mondays and Thursdays; thrice-weekly testing on Mondays, Wednesdays, and Fridays; and testing five times per week on each weekday. FIG. 5 shows the sensitivity of mcfDNA-seq by day prior to bloodstream infection using raw data without imputation. FIG. 6A-6D show the sensitivity of predictive mcfDNA-seq for bacterial BSI by frequency of testing. For each frequency of testing model, estimated sensitivities for event in each day of the week were plotted in a radar graph. Larger shaded area indicates higher overall sensitivity. The red line represents favorable sensitivity of 50%. Logical derivation was used to impute values for missing data. BSI, bloodstream infection; mcfDNA-seq, plasma microbial cell-free DNA sequencing. FIG. 6A shows the sensitivity for tests on Mondays. FIG. 6B shows the sensitivity for tests on Mondays and Thursdays. FIG. 6C shows the sensitivity for tests on Mondays, Wednesday, and Fridays. FIG. 6D shows the sensitivity for tests on Monday through Friday. Testing thrice weekly was required to keep minimum sensitivity above 50%. FIG. 7A-7D show the sensitivity of predictive mcfDNA-seq for bacterial BSI by frequency of testing using raw data without imputation. For each frequency of testing model, estimated sensitivities for event in each day of the week were plotted in a radar graph. Larger shaded area indicates higher overall sensitivity. BSI, bloodstream infection; mcfDNA-seq, plasma microbial cell-free DNA sequencing. FIG. 7A shows the sensitivity for tests on Mondays. FIG. 7B shows the sensitivity for tests on Mondays and Thursdays. FIG. 7C shows the sensitivity for tests on Mondays, Wednesday, and Fridays. FIG. 7D shows the sensitivity for tests on Monday through Friday.

Specificity was defined as the proportion of negative control samples in which no bacterial or fungal organisms were identified by mcfDNA-seq. Specificity for common BSI pathogens was an additional ad hoc measure defined as the proportion of negative control samples in which no common BSI pathogens were identified by mcfDNA-seq. Common BSI pathogens were defined as any member of a genus that comprised at least 1% of the organisms causing central line—associated BSI in children with cancer between August 2013 and December 2015, according to the previously described Children's Hospital Association Childhood Cancer & Blood Disorders Network BSI database. (Table 1). A 3rd degree penalized B-spline curve (PROC SGPLOT/PBSPLINE) was used to analyze possible trends in log 10(MPM+1) over time; for this analysis, all undetected organisms were assumed to have a DNA concentration of 0 MPM, but no other imputation was performed.

Results

This pilot cohort study included 47 pediatric patients with relapsed or refractory cancer. The causative pathogen was identified by mcf DNA-seq in the 3 days before onset of BSI in 12 of 16 episodes; of 33 negative control samples collected from the same patient population. mc.1DNA-seq was negative in 27 and identified no common pathogens in 30.

The primary outcomes were sensitivity of mcfDNA-seq for detecting a BSI pathogen during the 3 days before BSI onset and specificity of mcfDNA-seq in the absence of fever or infection in the preceding or subsequent 7 days.

Population and BSI Episodes

Between Aug. 9, 2017, and Jun. 4, 2018, 47 participants were enrolled (Table 2A) (27 (57%] male; median age [IQR], 10 [5-14] years). Nineteen BSI episodes occurred in 12 participants (3.3 per 1000 patient-days; 95% CI, 2-5.2 per 1000 patient days) (Table 2B) and predictive samples were available for 16 episodes, including 15 bacterial BSI episodes. Eight episodes (42%) were associated with signs of sepsis, including hypotension (n=3), requirement for urgent intervention (n=5), or intensive care unit admission (n=2). In the 3 days before the onset of infection, predictive sensitivity of mcfDNA-seq was 75% for all BSIs (12 of 16; 95% CI, 51%-90%) and 80% (12 of 15; 95% CI, 55%-93%) for bacterial BSIs. The specificity of mcfDNA-seq, evaluated on 33 negative control samples from enrolled participants, was 82% (27 of 33; 95% CI, 66%91%) for any bacterial or fungal organism and 91% (30 of 33; 95% CI, 76%-97%) for any common BSI pathogen, and the concentration of pathogen DNA was lower in control than predictive samples. A predictive period sample was available in 16 episodes. Broad-spectrum antibacterial therapy was administered during the prior week in 17 of 19 (89%) episodes, so a comparative subgroup analysis of the effect of pretreatment was not feasible.

TABLE 1
Exemplary organisms classified as common bloodstream
infection pathogens in children with cancer
Organism

Gram positive bacteria
Bacillus spp.¹
Clostridium spp.¹
Corynebacterium jeikeium
Enterococcus spp.¹
Lactobacillus spp.¹
Rothia spp.¹
Staphylococcus spp.¹
Streptococcus spp.¹
Gram negative bacteria
Citrobacter spp.¹
Escherichia coli¹
Klebsiella spp.¹
Pseudomonas spp.¹
Stenotrophomonas maltophilia¹
Fungi
Candida spp.¹

¹Identified in greater than 1% of episodes of central line associated bloodstream infection in children with cancer (Children's Hospital Association Childhood Cancer & Blood Disorders Network, August 2013-December 2015)

TABLE 2A
Exemplary characteristics of study participants

| Characteristic | No. (%) | |
|---|---|---|
| Total participants | 47 | (100) |
| Age, median (IQR), y | 10 | (5-14) |
| Sex | | |
| Male | 27 | (57) |
| Female | 20 | (43) |
| Race/ethnicity (self-reported) | | |
| White, non Hispanic | 19 | (40) |
| White Hispanic | 19 | (40) |
| Black | 5 | (11) |
| Other | 4 | (9) |
| Cancer type | | |
| Acute lymphoblastic leukemia | 22 | (47) |
| Acute myeloid leukemia | 23 | (49) |
| Other | 2 | (4) |
| Hematopoietic cell transplantation during study | 17 | (36) |
| Days on study, mean (SD) | 121 | (74) |

Abbreviation: IQR, interquartile range.

Sensitivity of mcfDNA-seq

The BSI pathogen was identified by mcfDNA-seq during the predictive period in 12 of 16 BSI episodes (predictive sensitivity, 75%; 95% CI, 51%-90%) and in 12 of 15 bacterial BSI episodes (predictive sensitivity, 80%; 95% CI, 55%-93%). Diagnostic sensitivity was 83% (15 of 18; 95% CI, 61%-94%); diagnostic sensitivity for bacterial BSI was 88% (15 of 17; 95% CI, 66%-97%). Blood cultures were collected during the week before BSI in 10 episodes (Table 4)

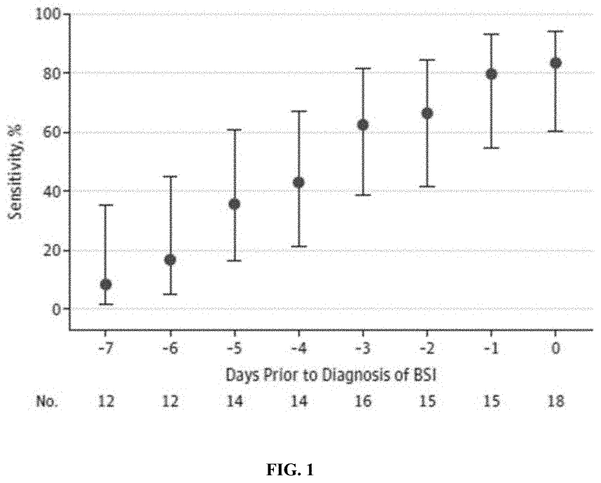

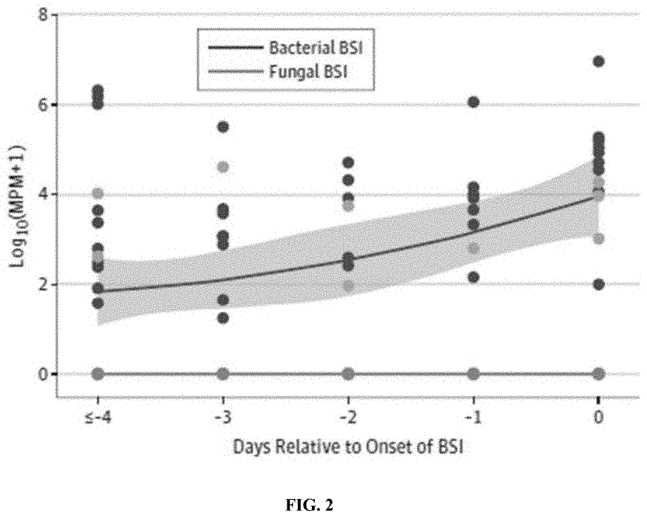

Daily predictive sensitivity of mcfDNA-seq for BSI is shown in FIG. 1. FIG. 1 shows the sensitivity of mcfDNA-seq for the prediction or diagnosis of BSI by day before the onset of infection. Logical derivation was used to impute values for missing data. BSI indicates bloodstream infection; mcfDNA-seq, plasma microbial cell-free DNA sequencing. Error bars show 95% CIs. Overall specificity of mcfDNA-seq was 82% (95% CI, 66%-91%), and specificity for common BSI pathogens was 91% (95% CI, 76%-97%). Pathogen-specific DNA concentrations typically increased in the days approaching BSI onset (FIG. 2 and Table 3). FIG. 2 shows the population kinetics of pathogen DNA by day before the onset of BSI. Circles represent individual values, lines represent penalized B-spline smoothing curves for bloodstream infection (BSI) episodes, and bands represent 95% CIs. Orange dots indicate a gram-negative pathogen; dark blue dots, a gram-positive pathogen; brown dots, overlapping samples. Assuming same-day results, projected median predictive sensitivity for bacterial BSI was 71% for twice-weekly testing (Table 5, FIG. 6A-6D and FIG. 7A-7D).

Specificity of mcfDNA-seq

Thirty-three negative control samples obtained from study participants underwent mcfDNA-seq testing. (Table 6); 27 of 33 had no bacterial or fungal organism identified (specificity, 82%; 95% CI, 66%-91%) and 30 of 33 had no common BSI pathogen identified (specificity, 91%; 95% CI, 76%-97%). The concentration of bacterial DNA in negative control samples was typically lower than in predictive samples, with a maximum of 609 MPM for any bacteria and a maximum of 112 MPM for common BSI pathogens compared with higher than 609 MPM in 11 of 16 predictive episodes (69%; 95% CI, 44%-86%) and higher than 112 MPM in 12 of 16 episodes (75%; 95% CI, 51%-90%).

Additional bacteria, including common pathogens, were identified by mcfDNA-seq in many samples collected before BSI episodes (Table 7). We attempted to assess the clinical significance of these, but all identified bacteria were potentially susceptible to empirical antimicrobial therapy, so treatment failure associated with untreated organisms was not evaluable. Fungal DNA was also identified by mcfDNA-seq in 2 participants with and 1 participant without evidence of invasive fungal infection.

TABLE 2B
Exemplary characteristics of the 19 included BSI episodes

| | | Diagnostic | Predictive | Neutro- |
|---|---|---|---|---|
| Episode | BSI Organism | Sample | Sample | penia |
| A | Escherichia coli (E. coli) | Yes | Yes | Yes |
| B | Staphylococcus | Yes | No | No |
| | epidermidis | | | |
| | (S. epidermidis) | | | |
| C | S. epidermidis | Yes | No | Yes |
| D | S. epidermidis | Yes | Yes | No |
| E | Enterococcus | Yes | Yes | No |
| | faecium (E. faecium) | | | |
| F | E. faecium | Yes | Yes | Yes |
| G | E. faecium | Yes | Yes | No |
| H | CoNS | Yes | Yes | Yes |
| I | Rothia mucilaginosa (R. | Yes | Yes | Yes |
| | mucilaginosa) | | | |
| J | S. epidermidis | Yes | Yes | Yes |
| K | Corynebacterium jeikeium | Yes | No | Yes |
| L | E. coli | Yes | Yes | Yes |
| M | E. coli & R. mucilaginosa* | Yes | Yes | Yes |
| N | E. coli | Yes | Yes | Yes |
| O | E. faecium | Yes | Yes | Yes |
| P | S. epidermidis | Yes | Yes | Yes |
| Q | Candida krusei | Yes | Yes | Yes |
| R | Enterococcus gallinarum | Yes | Yes | Yes |
| S | S. epidermidis | No | Yes | Yes |

Diagnostic sample, sample available from day of BSI onset; Predictive sample, sample available within 3 days before BSI onset; CoNS, unspeciated coagulase negative Staphylococcus sp.;
*polymicrobial infection consisting of 2 or more organisms grown from a single blood culture set.
Staphylococcus epidermidis and CoNS are Gram positive skin commensal organisms often identified as a cause of catheter associated bloodstream infections (Flygare, 2016, Genome Biol., 17:111);
Enteroccocus spp. are Gram positive gastrointestinal flora that are often associated with urinary, intrabdominal or catheter-associated bloodstream infections (Fung, 2018, Open Forum Infect Dis., 5:ofy301);
Rothia mucilaginosa is a Gram positive organism that commonly colonizes oropharyngeal mucosa and causes serious infections, including BSI and meningitis, in immunocompromised patients with mucositis (Ivy, 2018, J Clin Microbiol., 56:e00402-18);
Corynebacterium jeikeium is a Gram positive skin commensal organism that has been associated with serious infections in immunocompromised hosts including BSI, musculoskeletal infection and endocarditis.¹⁰

TABLE 3
Exemplary quantitative mcfDNA-seq data
for each bloodstream infection episode

| | | Max. MPM in | |
|---|---|---|---|
| BSI | Expected | predictive | MPM at BSI |
| Episode | organism(s) | period | onset |
| A | E. coli | 40,165 | 18,305 |
| B | S. epidermidis | N/A | 149,093 |
| C | S. epidermidis | N/A | 91,144 |
| D | S. epidermidis | 2,125 | 84,698 |
| E | E. faecium | 1,145,864 | 9,128,795 |
| F | E. faecium | 320,740 | 187,923 |
| G | E. faecium | 10,207 | 34,756 |
| H | CoNS | ND | ND |
| I | R. mucilaginosa | 8,015 | 9,213 |
| J | S. epidermidis | 20,515 | 157,456 |
| K | C. jeikeium | N/A | 98 |
| L | E. coli | ND | 9,280 |
| M | E. coli/R. | ND/ND | ND/ND |
| | mucilaginosa | | |
| N | E. coli | 616 | 1,023 |
| O | E. faecium | 4,760 | 11,443 |
| P | S. epidermidis | 7,890 | 52,495 |
| Q | C. krusei | ND | ND |
| R | E. gallinarum | 7,903 | 114,248 |
| S | S. epidermidis | 143 | N/A |

BSI, bloodstream infection; Max. MPM, maximal concentration of pathogen DNA in molecules per microliter; ND, not detected; N/A, sample not available.

TABLE 4
Exemplary blood cultures collected in the days before each bloodstream infection episode

| | Expected | Blood | | mcfDNA-seq |
|---|---|---|---|---|
| BSI Episode | organism(s) | culture day | Blood culture result | MPM |
| A | E. coli | 4 | Negative | 10,304 |
| B | S. epidermidis | None | N/A | |
| C | S. epidermidis | None | N/A | |
| D | S. epidermidis | None | N/A | |
| E | E. faecium | 6 | Negative | No sample |
| | | 4 | Negative | ND |
| | | 3 | Negative | No sample |
| | | 2 | Negative | ND |
| | | 1 | Negative | 1,145,864 |
| F | E. faecium | 5 | Negative | 1,476,869 |
| G | E. faecium | 3 | Negative | 753 |
| | | −1 | Negative | 10,207 |
| H | CoNS | −4 | Negative | No sample |
| | | −1 | Negative | ND |
| I | R. mucilaginosa | −6 | Negative | No sample |
| | | −5 | Negative | ND |
| | | −4 | Negative | ND |
| | | −3 | Negative | 17 |
| | | −2 | Negative | 256 |
| | | −1 | Negative | 8015 |
| J | S. epidermidis | None | N/A | N/A |
| K | C. jeikeium | None | N/A | N/A |
| L | E. coli | None | N/A | N/A |
| M | E. coli/R. mucilaginosa | −6 | Negative | ND |
| N | E. coli | None | N/A | N/A |
| O | E. faecium | None | N/A | N/A |
| P | S. epidermidis | −5 | S. epidermidis (1 of 2 lumens) | 80 |
| | | −3 | Negative | 1108 |
| Q | C. krusei | −6 | S. epidermidis (1 ot 2 lumens) | ND |
| | | −4 | Negative | ND |
| R | E. gallinarum | −1 | Negative | No sample |
| S | S. epidermidis | None | N/A | N/A |

BSI, bloodstream infection; None, no blood culture collected during the seven days prior to onset of BSI; CoNS, unspeciated coagulase negative Staphylococcus spp.; N/A, not applicable; mcfDNA-seq MPM, DNA concentration of the expected BSI pathogen by next generation metagenomic sequencing on day of blood culture; ND, expected BSI pathogen not detected by mcfDNA-seq.

TABLE 5
Exemplary effect of frequency of testing on sensitivity of mcfDNA-seq screening for bacterial BSI

| | Imputed data | | Raw data | |
|---|---|---|---|---|
| Testing frequency | Median sensitivity | (Range) | Median sensitivity | (Range) |
| Mondays | 46.2% | (9.1%, 85.7%) | 55.6% | (33.3%, 80%) |
| Mondays & Thursdays | 71.4% | (46.2%, 85.7%) | 72.7% | (55.6%, 80%) |
| Mondays, Wednesdays & Fridays | 71.4% | (66.7%, 85.7%) | 72.7% | (69.2%, 80%) |
| Monday through Friday | 85.7% | (66.7%, 85.7%) | 80% | (69.2%, 80%) |

TABLE 6
Exemplary plasma microbial cell-free DNA sequencing results for
bacteria in negative control samples from study participants

| Control | | | | Common BSI |
|---|---|---|---|---|
| Sample | Neutropenia | Organisms Identified | MPM | Pathogen |
| 1 | No | Klebsiella pneumoniae | 112 | Yes |
| | | Clostridium perfringens | 99 | Yes |
| 2 | No | None | | No |
| 3 | Yes | None | | No |
| 4 | No | None | | No |
| 5 | Yes | None | | No |
| 6 | No | None | | No |
| 7 | Yes | Veillonella parvula | 41 | No |
| 8 | Yes | None | | No |
| 9 | No | None | | No |
| 10 | Yes | None | | No |
| 11 | Yes | None | | No |
| 12 | No | None | | No |
| 13 | Yes | None | | No |
| 14 | Yes | None | | No |
| 15 | Yes | Finegoldia magna | 129 | No |
| 16 | Yes | None | | No |
| 17 | No | None | | No |
| 18 | Yes | None | | No |
| 19 | Yes | None | | No |
| 20 | Yes | None | | No |
| 21 | No | Lactobacillus fermentum | 25 | Yes |
| | | Helicobacter pylori | 38 | No |
| 22 | Yes | None | | No |
| 23 | Yes | Neisseria sicca | 609 | No |
| 24 | Yes | None | | No |
| 25 | Yes | None | | No |
| 26 | Yes | None | | No |
| 27 | No | None | | No |
| 28 | Yes | Escherichia coli | 85 | Yes |
| 29 | No | None | | No |
| 30 | No | None | | No |
| 31 | Yes | None | | No |
| 32 | Yes | None | | No |
| 33 | Yes | None | | No |

MPM, concentration of DNA in molecules per microliter.

TABLE 7
Exemplary additional bacterial organisms identified during bloodstream infection episodes

| BSI | Expected | | Max. | Effective | Antibiotic |
|---|---|---|---|---|---|
| Episode | orsanism(s) | Additional identified organism | MPM | antibiotic | days |
| A | E. coli | None | N/A | N/Λ | N/A |
| B | S. epidermidis | None | N/A | N/A | N/A |
| C | S. epidermidis | E. faecalis | 2,006 | Vancomycin | 5 |
| D | S. epidermidis | C. difficile | 5,019 | Vancomycin | 4 |
| E | E. faecium | S. epidermidis | 763,462 | Vancomycin | 3 |
| | | S. thermophilus | 317 | Vancomycin | 3 |
| | | L. gasseri | 2,000 | Vancomycin | 3 |
| F | E. faecium | S. epidermidis | 509,208 | Vancomycin | 14 |
| G | E. faecium | Alloiococcus otitis | 743 | Ampicillin | 7 |
| | | H. parainfluenzae | 1,472 | Cefepime | 4 |
| | | L. gasseri | 534 | Vancomycin | 4 |
| | | Staphylococcus auricularis | 1,273 | Vancomycin | 4 |
| | | Streptococcus oralis | 1,411 | Vancomycin | 4 |
| | | Turicella otitidis | 454 | Vancomycin | 4 |
| H | CoNS | E. faecium | 8,997 | Vancomycin | 6 |
| | | H. parainfluenzae | 4,979 | Cefepime | 3 |
| | | L. acidophilus | 81,508 | Vancomycin | 6 |
| | | L. casei | 52,189 | Penicillin VK | 4 |
| | | S. thermophilus | 161,209 | Vancomycin | 6 |
| I | R. mucilaginosa | S. thermophilus | 18 | Vancomycin | 9 |
| | | S. mitis | 47,369 | Vancomycin | 9 |
| | | E. faecium | 150 | Vancomycin | 9 |
| J | S. epidermidis | S. mitis | 1,091 | Vancomycin | 10 |
| K | C. jeikeium | S. epidermidis | 182 | Vancomycin | 3 |
| | | Campylobacter showae | 46 | Meropenem | 4 |
| | | H. parainfluenzae | 42 | Meropenem | 4 |
| | | Pseudomonas pseudoalcaligenes | 37 | Meropenem | 4 |
| L | E. coli | Kingella oralis | 67 | Ceftriaxone | 1 |
| | | Solobacterium moorei | 68 | Meropenem | 10 |
| | | S. epidermidis | 969 | levofloxacin | 8 |
| M | E. coli, R. mucilaginosa | S. mitis | 698 | Cefepime | 15 |
| | | Granulicatella adiacens | 28 | Vancomycin | 12 |
| N | E. coli | Agrobacterium tumefaciens | 65 | Vancomycin | 6 |
| O | E. faecium | F. nucleatum | 5,470 | Linezolid | 11 |
| | | H. parainfluenzae | 2,476 | Cefepime | 4 |
| P | S. epidermidis | S. capitis | 47 | Vancomycin | 10 |
| Q | C. krusei | S. capitis | 47 | Vancomycin | 10 |
| R | E. gallinarum | F. nucleatum | 465 | Metronidazole | 11 |
| | | Prevotella melaninogenica | 220 | Metronidazole | 11 |
| | | L. rhamnosus | 2,840 | Linezolid | 5 |
| S | S. epidermidis | S. mitis | 48 | Vancomycin | 9 |
| | | Actinomyces graevenitzii | 73 | Vancomycin | 9 |
| | | P. melaninogenica | 73 | Cefepime | 5 |
| | | S. thermophilus | 138 | Vancomycin | 9 |

Discussion

In patients with imminent BS, it appears that mcfDNA-seq can identify clinically relevant pathogens days before onset of attributable symptoms. A clinically relevant pathogen can be identified by mcfDNA-seq days before the onset of BSI in a majority of episodes, potentially enabling preemptive treatment. Clinical application appears feasible pending further study.

This prospective pilot study shows that mcfDNA-seq has the potential to predict most episodes of BSI before onset in high-risk pediatric cancer patients. The estimated predictive sensitivity of 75% (95% CI, 51%-90%) exceeds the predefined favorable value of 50%, which was chosen because it represents the approximate efficacy of antibacterial prophylaxis. In addition to BSI organisms, viruses and invasive fungi that infect immunocompromised patients were also detected. Further studies are needed to determine whether mcfDNA-seq can reliably predict infection with nonbacterial pathogens in this patient population.

Limitations

A specificity of 82% would make mcfDNA-seq screening impractical because of the high false-positive rate; specificity might be improved by excluding uncommon BSI pathogens, applying quantitative break points, or performing short interval repeat testing. Return of mcfDNA-seq results in a time frame that allows implementation of screening may not yet be feasible in most centers. Technological and practical advances will be required to reduce turnaround time considerably and might also improve sensitivity to predict BSI even earlier. Application of mcfDNA-seq screening to febrile neutropenia and clinically documented and other microbiologically documented infections was not evaluated in this study and will be important for future studies. Once these challenges are overcome, implementation of predictive mcfDNA-seq has the potential to significantly reduce treatment-related morbidity and mortality of pediatric cancer patients and could potentially be applied to other immunocompromised patient populations.

Conclusions

We provide to our knowledge the first evidence that mcfDNA-seq can predict infections in approximately 75% of relapsed pediatric cancer patients with impending BSI with a specificity of more than 80%. Strategic implementation and continued technological advancements may enable the use of mcfDNA-seq to guide preemptive therapy and reduce infection-related morbidity and mortality in high-risk immunocompromised patients.

TABLE 8
Exemplary characteristics of invasive fungal infections
Characteristics of Invasive Fungal Infections

| | | | | | |
|---|---|---|---|---|---|
| | | Causative | Diagnosis | Predictive | Prediction |
| Episode | Site | Organism | NGS+ | NGS+ | Day |

Proven

| | | | | | |
|---|---|---|---|---|---|
| PQ109 #1 | Paranasal Sinus | Alternaria alternata | No* | Yes | −21 |
| PQ118 | Paranasal Sinus | Mucor velutinosus | Yes | Yes | −10 |
| PQ120 | Gingiva | Aspergillus flavus/oryzae | Yes | Yes | −15 |
| PQ181 | Paranasal Sinus | Curvularia sp. | No* | No* | |

Probable

| | | | | | |
|---|---|---|---|---|---|
| PQ107 | Disseminated | Histoplasma capsulatum | Yes | Yes | −29 |
| PQ109 #2 | Pulmonary | Aspergillus sp. | No | No | |
| PQ123 | Pulmonary | Aspergillus flavus/oryzae | Yes | Yes | −15 |
| PQ175 | Pulmonary | Aspergillus sp. | No | No | |

Possible

| | | | | |
|---|---|---|---|---|
| PQ124 | Pulmonary | Unknown | No | No |
| PQ147 | Pulmonary | Unknown | No | No |
| PQ174 | Pulmonary | Unknown | No | No |

“Suspected”

| | | | | |
|---|---|---|---|---|
| PQ180 | Pulmonary | Unknown | No | No |

Diagnosis NGS+, Positive test ≤1 day of diagnosis;
Predictive NGS+, Positive test >1 day before diagnosis;
Prediction Day, Day specific fungal DNA first identified by mcfDNANGS;
*The fungal DNA was identified only by manual review of sequence data.

### Example 2

2.1 Background/Introduction

Diagnosis of invasive fungal infections (IFIs), a life-threatening complication of cancer therapy or hematopoietic cell transplantation (HCT) can be invasive and challenging, and IFI has poor outcomes. Prediction or early non-invasive diagnosis of IFI in high-risk hosts before onset of symptoms could reduce morbidity and mortality.

2.1.1 Objective

Because non-invasive plasma mcfDNA NGS can detect invasive fungal infections, and may predict bloodstream infections in immunocompromised patients, we hypothesized that mcfDNA NGS might also predict invasive fungal infection before clinical presentation.

2.2 Methods

In a prospective study, serial remnant plasma samples were collected from pediatric patients undergoing treatment for relapsed or refractory leukemia. All samples collected within 30 days before clinical diagnosis of non-fungemic IFI were tested for fungal DNA by mcfDNA NGS using clinical and research assays by Karius, Inc.; because of overlapping clinical syndromes, non-fungal DNA was not considered in this study.

2.3 Results

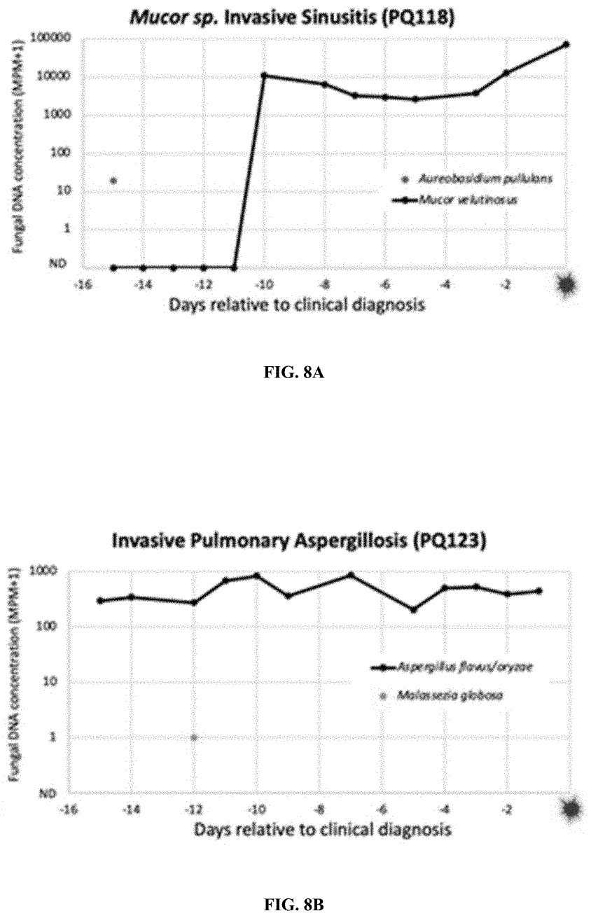

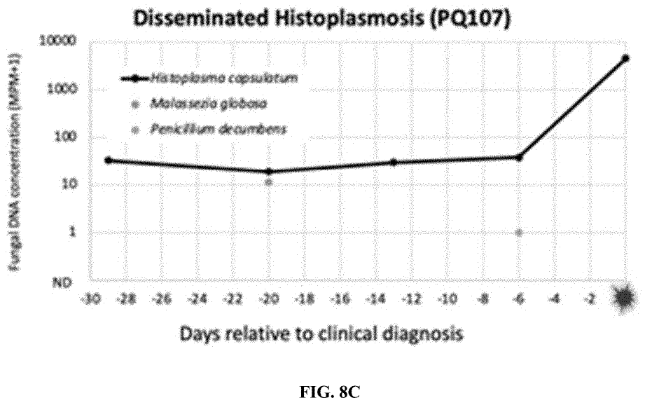

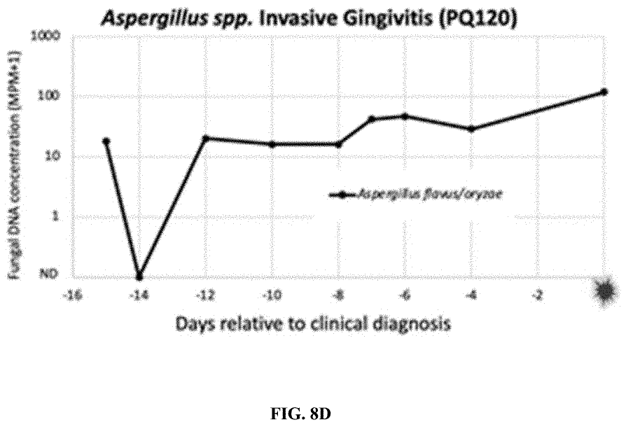

There were 12 episodes of IFI in 11 participants with >1 sample available from both diagnostic (within 1 day of diagnosis) and predictive (2 to 28 days prior to diagnosis) periods (1 “suspected” and 3 possible, 4 probable, and 4 proven by EORTC definitions). Of 8 probable or proven IFIs, 4 (50%) had a relevant fungal pathogen identified mcfDNA NGS at diagnosis. In each of these, and one additional case, the fungal DNA was also detectable prior to clinical onset of IFI (Range 10 to 29 days; FIG. 8A-8D). FIG. 8A shows Mucor velutinosus DNA kinetics in samples collected prior to invasive fungal infection in a child (PQ118) with leukemia. Pathogen DNA concentration is given in molecules of fungus per microliter of plasma (MPM) vs. days relative to clinical diagnosis of invasive sinusitis. Also given is a single measurement of a non-infective fungus, Aureobosidium pullulans, present in, at day −15 the sample. FIG. 8B shows Aspergillus flavus/oryzae DNA kinetics in samples collected prior to invasive pulmonary Aspergillosis infection in a child (PQ123) with leukemia. Pathogen DNA concentration is given in molecules of fungus per microliter of plasma (MPM) vs. days relative to clinical diagnosis of an invasive pulmonary infection. Also given is a single measurement of a non-infective fungus, Malassezia globosa, present in the sample at day −12. FIG. 8C shows Histoplasma capsulatum DNA kinetics in samples collected prior to invasive disseminated Histoplasmosis infection in a child (PQ107) with leukemia. Pathogen DNA concentration is given in molecules of fungus per microliter of plasma (MPM) vs. days relative to clinical diagnosis of invasive infection. Also given are single measurements of a non-infective fungus, Malassezia globosa, present in the sample at day −20 and Penicillium decumbens, present in the sample at day −6. FIG. 8D shows Aspergillus flavus/oryzae DNA kinetics in samples collected prior to invasive pulmonary Aspergillus spp. invasive gingivitis infection in a child (PQ120) with leukemia. Pathogen DNA concentration is given in molecules of fungus per microliter of plasma (MPM) vs. days relative to clinical diagnosis of invasive gingivitis. In an additional case, manual review of sequence data identified the fungal DNA at diagnosis and intermittently during the prior month. Of 4 suspected or possible IFI, no fungal pathogens were identified at diagnosis.

2.4 Conclusion

mcfDNA NGS can identify fungal pathogen DNA before clinical onset of IFI, so might predict IFI in immunocompromised hosts, and may differentiate fungal infection from other etiologies of lung nodules or infiltrates.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating a subject at risk for a bloodstream infection comprising:
a. collecting one or more blood samples from the subject, wherein the one or more blood samples comprise microbial cell-free nucleic acids (mcfNA) and the subject is afebrile or blood-culture negative;
b. detecting an amount of a pathogen associated with the bloodstream infection based on the microbial cell-free nucleic acids (mcfNA) in the one or more blood samples;
c. predicting that the subject at risk for the bloodstream infection will experience a sign or symptom of a bloodstream infection based on the amount of the pathogen associated with the bloodstream infection, wherein the predicting has a predictive sensitivity of at least 75% for samples collected at least three days prior to onset of the sign or symptom of the bloodstream infection; and
d. administering a therapeutic treatment to the subject prior to onset of the symptom of the bloodstream infection.

2. The method of claim 1, wherein the pathogen associated with the bloodstream infection is a bacterium, and the bloodstream infection is a bacterial bloodstream infection.

3-4. (canceled)

5. The method of claim 1, wherein the pathogen associated with the bloodstream infection is a fungus and the bloodstream infection is a fungal infection.

6. The method of claim 5, wherein the fungus is a Candida spp.

7. The method of claim 5, wherein the fungus is a Candida krusei.

8. The method of claim 1, wherein the mcfNA are microbial cell-free DNA.

9. The method of claim 1, wherein the one or more blood samples are one or more plasma samples.

10-11. (canceled)

12. The method of claim 1, wherein the predictive sensitivity of at least 75% is a predictive sensitivity of at least 85% for a bacterial bloodstream infection.

13-15. (canceled)

16. The method of claim 1, wherein the therapeutic treatment is a pathogen-directed therapy.

17. The method of claim 16, wherein the pathogen-directed therapy is at least one therapy selected from the group consisting of: vancomycin, ampicillin, cefepime, penicillin, meropenem, ceftriaxone, levofloxacin, and linezolid.

18. The method of claim 1, wherein the subject has no infection within seven days prior to collecting the one or more blood samples.

19-23. (canceled)

24. The method of claim 1, wherein the subject is blood culture negative during the collecting of the one or more blood samples.

25-30. (canceled)

31. A method of processing and analyzing a blood sample from a blood cancer patient who is asymptomatic for an infection by a bacterial or fungal microbe or blood-culture negative for the bacterial or fungal microbe comprising:
a. preparing at least one plasma sample comprising microbial cell-free nucleic acids (mcfNA) from the blood cancer patient asymptomatic for the infection by the bacterial or fungal microbe or blood-culture negative for the bacterial or fungal microbe;
b. preparing a first library comprising the mcfNA attached to adapters;
c. subjecting the first library comprising the mcfNA attached to the adapters to next generation sequencing to produce sequence reads;
d. aligning the sequence reads to bacterial or fungal DNA sequences in a reference data set to obtain aligned sequence reads; and
e. detecting a presence of and quantifying the bacterial or fungal organism at a species or strain level based on the aligned sequence reads.

32. The method of claim 0, wherein the microbial cell-free nucleic acids comprise microbial cell-free DNA.

33-38. (canceled)

39. The method of claim 31, further comprising spiking the at least one plasma sample with a known concentration of synthetic DNA molecules.

40. The method of claim 31, wherein the blood cancer is a leukemia.

41. (canceled)

42. The method of claim 31, wherein the patient is receiving chemotherapy.

43. The method of claim 31, wherein the patient is the recipient of a hematopoietic stem cell transplant.

44-48. (canceled)

49. The method of claim 31, wherein the infection by a bacterial or fungal microbe is a bacterial bloodstream infection.

50. (canceled)

51. The method of claim 31, wherein the infection by a bacterial or fungal microbe is an invasive fungal infection.

52. (canceled)

53. A method of processing and analyzing a blood sample from a cancer patient at risk of a bacterial or fungal infection comprising:
a. preparing plasma samples comprising microbial cell-free DNA (mcfDNA) from at least two longitudinal blood samples collected from the cancer patient within seven days prior to the onset of a bloodstream infection;
b. isolating the mcfDNA from the plasma samples;
c. attaching adapters to the mcfDNA to produce a DNA library comprising mcfDNA attached to the adapters;
d. obtaining sequence reads from the DNA library;
e. aligning the sequence reads to bacterial or fungal DNA sequences in a reference data set to obtain aligned sequence reads;
f. identifying the infection by a bacterial or fungal organism based on the aligned sequence reads; and
g. quantifying the mcfDNA at the species or strain level prior to onset of the infection by a bacterial or fungal organism.

54-70. (canceled)

71. The method of claim 1, wherein the onset of infection is the onset of an infection that is blood-culture positive.
